(19) United States
(12) Patent Application Publication
(10) Pub. No.: US 2024/0085404 A1
(43) Pub. Date: Mar. 14, 2024

(54) IN VITRO CELL-BASED ASSAY FOR PREDICTING PHARMACOKINETICS AND BRAIN PENETRATION OF BIOLOGICS

(71) Applicant: Genentech, Inc., South San Francisco, CA (US)

(72) Inventors: Chang LIU, Belmont, CA (US); John Hok Nin LOWE, Dublin, CA (US); Shan CHUNG, Maple Valley, WA (US)

(73) Assignee: Genentech, Inc., South San Francisco, CA (US)

(21) Appl. No.: 18/486,325

(22) Filed: Oct. 13, 2023

Related U.S. Application Data
(63) Continuation of application No. PCT/US2022/024345, filed on Apr. 12, 2022.
(60) Provisional application No. 63/187,750, filed on May 12, 2021.
(60) Provisional application No. 63/174,913, filed on Apr. 14, 2021.

(51) Int. Cl. G01N 33/50; G01N 33/68

(52) U.S. Cl. CPC G01N 33/5044; G01N 33/6854; G01N 2333/70535

(57) ABSTRACT

The present disclosure relates to methods and compositions useful for measuring the transcytosis or recycling of a molecule. In particular, the present disclosure relates to in vitro receptor-dependent transcytosis or recycling assays for evaluating the clearance rates of therapeutic antibody molecules and Fc-fusion proteins in humans and animals.

### CROSS-REFERENCE TO RELATED APPLICATIONS

This application is a continuation of PCT/US2022/024345, filed Apr. 12, 2022, which claims the benefit of U.S. Provisional Application No. 63/174,913, filed Apr. 14, 2021, and of U.S. Provisional Application No. 63/187,750, filed May 12, 2021, the contents of which are incorporated herein by reference in their entireties.

### SEQUENCE LISTING

This application contains a Sequence Listing which has been submitted electronically in XML format and is hereby incorporated by reference in its entirety. Said XML copy, created on Oct. 5, 2023, is named P36572-US-2.xml and is 9,696 bytes in size.

### FIELD OF INVENTION

The present disclosure relates to methods and compositions useful for measuring the recycling of a molecule by a cell layer. In particular, the present disclosure relates to in vitro receptor-dependent recycling assays, and recycling and transcytosis assays, for evaluating in vivo pharmacokinetic profiles (e.g., clearance rates, and/or half-lives) and tissue penetration of molecules that interact with FcRn, e.g., therapeutic antibodies, Fc fusion molecules, and molecules linked to albumin.

### BACKGROUND

Therapeutic monoclonal antibodies (mAbs) have become a major class of pharmaceutical products worldwide due to their proven effectiveness in the treatment of a variety of diseases and their desirable pharmacological properties. One of the favorable pharmacological properties of mAb drugs is their typically long circulating half-life. The extended half-life of biotherapeutic products could potentially allow for less frequent dosing and/or lower dose of the drug, which may reduce cost of care, improve patient compliance, and/or reduce concentration-dependent cytotoxicity/adverse events.

Significant progress has been made in understanding pharmacokinetics (PK) profiles of mAb drugs in animals and humans. Whereas many mAb drugs exhibit similar PK behavior that is analogous to endogenous IgG, substantial heterogeneity in non-specific clearance rate of mAb drugs in humans is still commonly observed. Therefore, evaluation and selection of clinical candidates for desirable PK properties is an important early step during drug development. A broad range of technologies involving in silico, in vitro and in vivo analyses have been employed to evaluate and/or predict PK behavior of mAbs in humans (Dostalek et al., 2017, MAbs 9 (5):756-766). However, translation of PK data from animal to humans, and from in vitro assays to in vivo readouts, remains elusive to drug developers (Vugmeyster et al., 2012, World J Biol Chem 3(4): 73-92). In addition, while some animal models such as human FcRn transgenic mice (Avery et al., 2016, MAbs, 8(6), 1064-1078) and non-human primate (Deng et al., 2011, Drug Metab Dispos 38(4): 600-605) have been successfully used to project human PK of mAbs, these studies are typically time-consuming, costly, and involve animal sacrifice.

There are many complexities in predicting human PK of mAbs from animal studies and in vitro assays. Target-mediated drug disposition (TMDD) is known to impact dose-dependent PK behavior of mAbs resulting in nonlinear distribution and elimination. In the absence of TMDD, mAbs are expected to exhibit linear elimination and non-specific clearance rate which reflects target-independent molecule-specific drug catabolism. The non-specific clearance of mAbs is mediated mostly by lysosomal degradation in the reticuloendothelial system where the neonatal Fc receptor (FcRn)-mediated savage pathway plays a major role. Structurally, FcRn is a heterodimer composed of a transmembrane heavy chain (FCGRT) homologous to major histocompatibility complex (MHC) class-I like molecules and a soluble light chain, β2 microglobulin (B2M). FcRn binds to the Fc domain of endogenous IgG at acidic pH (less than 6.5), but only minimally at neutral or basic pH (greater than 7.0). This unique property allows FcRn to protect Fc-containing molecules from degradation by binding to them in acidic endosomes after their uptake into cells and then transport them back to the cell surface and release them to the circulation at physiological pH. In contrast, internalized molecules that are not bound to FcRn are directed to lysosomes for degradation (see FIG. 1 and Roopenian et al., Nat Rev Immunol. 2007 September; 7(9):715-25).

While the contribution of FcRn in prolonging half-lives of Fc-containing proteins is well recognized (Roopenian et al., 2003, J Immunol, 170(7), 3528-3533), reports of a lack of correlation between FcRn binding and PK for mAb drugs in human (Suzuki, 2010; Zheng, 2011; Hotzel, 2012) suggests that binding affinity to FcRn is not the sole determinant for the elimination rate of Fc-containing proteins. Given the function of FcRn in intracellular trafficking, the dynamics of endosomal sorting and trafficking of Fc-containing molecule/FcRn complexes are likely to impact overall efficiency of the FcRn-mediated salvage mechanism and hence the molecule's non-specific clearance rate. In addition, the processes of cellular uptake via pinocytosis or endocytosis as well as degradation in lysosome could also play a role in determining the rate and extent of mAb degradation (Junghans & Anderson, 1996, PNAS 93(11), 5512-5516). Lastly, biochemical characteristics such as charge/pI, hydrophobicity, nonspecific binding, and other factors have been reported to impact mAb disposition (Datta-Mannan et al., 2015, MAbs 7(3), 483-493; Igawa et al., 2010 Protein Eng Des Sel 23(5), 385-392; Sharma et al., 2014 PNAS 111(52); Hotzel et al., 2012 MAbs 4(6), 753-760). These molecular characteristics may in themselves influence mAb structure so as to alter the “true” interaction with FcRn in vivo or may contribute to nonspecific cell surface binding that alters the mode/rate of internalization and the relative proportion of FcRn-mediated intracellular trafficking pathways including recycling and transcytosis.

In vivo animal studies performed in rodents and nonhuman primates are commonly used to predict PK of therapeutic antibodies in humans (Avery et al., MAbs, 2016, 8(6), 1064-1078; Deng et al., MAbs 2011, 3(1), 61-66). These approaches involve sacrificing animals, are less cost-effective, and cannot be conducted in a high throughput manner. Based on empirical scaling methods, in some cases PK in animal studies could not faithfully predict that in human and demonstrated cross-species difference in PK, likely due to the species difference in FcRn binding, target-mediated drug disposition, and triggering of differential immune responses (Wang et al., 2016, Biopharm Drug Dispos, 37(2), 51-65). Efforts on in vitro assays have been employed to evaluate PK with less time- and cost-consuming and animal-sparing approaches. Most of these methodologies take into consideration one or a subset of molecular properties that may affect PK, such as non-specific binding (Hotzel et al., MAbs 2012, 4(6), 753-760). FcRn interactions (Schlothauer et al., MAbs 2013, 5(4), 576-586; Souders et al., MAbs 2015, 7(5), 912-921). and binding to Heparin or extra-cellular matrix (de Ridder et al., Cerebrovasc Dis 2013, 35(1), 60-63; Kraft et al., MAbs 2020, 12(1)). These methods usually depend on a cutoff threshold, sometimes showed limited success in a direct PK prediction and had to combine with another method to improve predictive accuracy.

The lack of vitro models for real-world behavior of antibodies is a particular challenge in the context of brain penetration. Central nervous system (CNS) disorders are a serious health burden, and there has been strong drive in developing effective therapies for CNS disorders for many decades. The blood brain barrier, a highly specialized tissue comprising endothelial cells (among others) with tight junctions, regulates the passage of molecules into the brain, including preventing the transport of the vast majority of current therapeutics from the blood stream into the CNS. While there are different methods to modify the brain penetration of molecules, it is highly challenging to apply these methods in preclinical drug development without robust models to evaluate their effectiveness. Due to poor correlation between existing models and in vivo outcomes, brain penetration of potential therapeutics often has to be tested empirically, which is time consuming and expensive.

A human microvascular endothelial cell (HMEC1) line that modified to express human FcRn has been developed and evaluated, and the recycling/residual mAb ratio was found to correlate with their serum half-lives in human FcRn transgenic mice (Grevys et al., Nat Commun 2018, 9(1), 621). However, the dataset was limited to engineered IgG with much enhanced FcRn binding affinity, but did not include normal IgG framework that was more commonly used in therapeutic antibodies. In addition, the cells were cultured in regular cell culture plates which may not ideal for cell polarization and may result in contamination of transcytosis component into the recycling samples.

There thus remains a need for methods to predict the in vivo PK properties (e.g., clearance, half-life, or others), and brain penetration, of therapeutic molecules that can be conducted in a short amount of time and at reasonable cost to aid in evaluation and selection of therapeutic molecules.

### SUMMARY OF THE INVENTION

The present disclosure relates to methods, assays, assay systems, kits and compositions useful for measuring the movement or transport of a molecule of interest to and from a cellular membrane, including the recycling of a molecule of interest, and the recycling and transcytosis of a molecule of interest. In particular, the present disclosure provides in vitro receptor-dependent transcytosis and recycling assays for evaluating in vivo pharmacokinetic profiles (including, e.g., in vivo clearance (CL), volume of distribution (Vd), area under the curve (AUC), bioavailability, maximum/minimum plasma concentrations (Cmax/Cmin) of molecules that interact with molecular transport receptors (e.g., FcRn), including molecules such as therapeutic antibodies, Fc fusion molecules, and molecules linked to albumin. The methods and assays provided herein may, compared to other methods, better encompass multiple factors that may affect in vivo PK behavior of test molecules (including antibodies), such as nonspecific binding and interactions with cell surface, pH-dependent binding (e.g., FcRn binding) and intracellular trafficking pathways. The methods and assays provided herein may, compared to other methods, better predict and/or model in vivo tissue penetrance (such as brain penetrance) of a molecule of interest (including antibodies). The provided methods and assays may further provide improved apico-basolateral polarization of the cells, and/or clearer evaluation of recycling vs transcytosis components, in comparison to other methods and/or assays.

In certain embodiments, the present disclosure is directed to methods for determining the recycling of a plurality of molecules. For example, the determining can comprise introducing the plurality of molecules into a first chamber, wherein: the first chamber is separated from a second chamber by a cell layer, and the first and second chambers comprise an aqueous solution; incubating the plurality of molecules in the first chamber; replacing the aqueous solution in both the first and second chambers; and measuring the amount of the plurality of molecules that is released from the cell layer into the first chamber; wherein the aqueous solution is at physiological pH, and the cell layer comprises cells that express a receptor that mediates molecular transport.

In certain embodiments, the method further comprises measuring the transcytosis of the plurality of molecules across the cell layer. In certain embodiments, measuring the transcytosis comprises, after the aqueous solution has been replaced, measuring the amount of the plurality of molecules in the second chamber.

In certain embodiments, the method comprises incubating the plurality of molecules in the first chamber in the presence of an agent, and determining whether the agent affects the recycling of the plurality of molecules.

In certain embodiments, the present disclosure is directed to a method of determining the tissue penetrance of a plurality of molecules, comprising: a) introducing the plurality of molecules into a first chamber, wherein the first chamber is separated from a second chamber by a cell layer, and the first and second chambers comprise an aqueous solution; wherein the aqueous solution is at physiological pH, and the cell layer comprises cells that express a receptor that mediates molecular transport; b) measuring the amount of the plurality of molecules that is recycled from the first chamber into the cell layer and back to the first chamber; c) measuring the amount of the plurality of molecules that is transcytosed from the first chamber to the second chamber; and d) determining the tissue penetrance of the plurality of molecules based on the ratio of transcytosed to recycled molecules. In some embodiments, the tissue penetrance is brain penetrance.

In certain embodiments, measuring the plurality of molecules that is recycled comprises: after introducing the plurality of molecules into the first chamber, incubating the plurality of molecules in the first chamber; replacing the aqueous solution in both the first and second chambers; and measuring the amount of the plurality of molecules that is released from the cell layer into the first chamber.

In certain embodiments, measuring the plurality of molecules that is transcytosed comprises: after introducing the plurality of molecules into the first chamber, incubating the plurality of molecules in the first chamber; replacing the aqueous solution in both the first and second chambers; and measuring the amount of the plurality of molecules that is released from the cell layer into the second chamber.

In certain embodiments, measuring the amount of the plurality of molecules that is recycled and measuring the amount of the plurality of molecules that is transcytosed independently comprise the use of an enzyme-linked immunosorbent assay (ELISA), liquid-scintillation counting (LSC), quantitative PCR, a fluorescence reader system, mass spectrometry, or any combinations thereof In certain embodiments, the tissue penetrance is brain penetrance.

In certain embodiments, the method comprises incubating the plurality of molecules in the first chamber in the presence of an agent, and determining whether the agent affects the tissue penetrance of the plurality of molecules.

In certain embodiments, the present disclosure is directed to a method of determining a pharmacokinetic (PK) parameter of a plurality of molecules, comprising: a) introducing the plurality of molecules into a first chamber, wherein the first chamber is separated from a second chamber by a cell layer, and the first and second chambers comprise an aqueous solution; wherein the aqueous solution is at physiological pH, and the cell layer comprises cells that express a receptor that mediates molecular transport; b) measuring the amount of the plurality of molecules that is recycled from the first chamber into the cell layer and back to the first chamber; and c) determining the PK parameter based on the amount of the plurality of molecules that is recycled.

In certain embodiments, measuring the plurality of molecules that is recycled comprises: after introducing the plurality of molecules into the first chamber, incubating the plurality of molecules in the first chamber; replacing the aqueous solution in both the first and second chambers; and measuring the amount of the plurality of molecules that is released from the cell layer into the first chamber.

In certain embodiments, the cells express a heterologous FcRn. In certain embodiments, measuring the amount of the plurality of molecules that is recycled comprises the use of an enzyme-linked immunosorbent assay (ELISA), liquid-scintillation counting (LSC), quantitative PCR, a fluorescence reader system, or mass spectrometry.

In certain embodiments, the PK parameter is a measure of in vivo clearance, volume of distribution, area under the curve (AUC), bioavailability, or in vivo half-life of the plurality of molecules.

In certain embodiments, the method comprises incubating the plurality of molecules in the first chamber in the presence of an agent, and determining whether the agent affects the PK parameter of the plurality of molecules.

In certain embodiments, the plurality of molecules is a plurality of a single molecule. In certain embodiments, the plurality of molecules is a plurality of distinct molecules. In certain embodiments, the cell layer comprises a cell monolayer. In certain embodiments, the receptor that mediates molecular transport is a receptor that mediates intracellular transport of molecules. In certain embodiments, the receptor that mediates molecular transport is transferrin receptor, an Fc receptor, megalin, or cubulin. In certain embodiments, the receptor that mediates molecular transport is a neonatal Fc receptor (FcRn).

In certain embodiments, the cells are eukaryotic cells, mammalian cells or kidney cells. In certain embodiments, the cells are Madin-Darby Canine Kidney (MDCK) cells.

In certain embodiments, measuring the amount of the plurality of molecules that is released from the cell layer comprises the use of an enzyme-linked immunosorbent assay (ELISA), liquid-scintillation counting (LSC), quantitative PCR, a fluorescence reader system, or mass spectrometry.

In certain embodiments, the plurality of molecules is incubated in the first chamber from about 1 hour to about 48 hours. In certain embodiments, the plurality of molecules is incubated in the first chamber from about 1 hour to about 30 hours. In certain embodiments, replacing the aqueous solution comprises washing the first chamber. In certain embodiments, the method further comprises incubating the first and second chambers with the replacement aqueous solution prior to measuring. In certain embodiments, the first and second chambers are incubated with the replacement aqueous solution for between 1 hour and 6 hours prior to measuring. In certain embodiments, the incubation is at a temperature of between about 35° C. to about 39° C.

In certain embodiments, the plurality of molecules are Fc-containing molecules. In certain embodiments, the Fc-containing molecules are receptor Fc fusion molecules. In certain embodiments, the plurality of molecules are antibodies. In certain embodiments, the antibodies are monoclonal antibodies. In certain embodiments, the FcRn is selected from the group consisting of human RcRn, mouse FcRn, rat FcRn, and cynomolgus FcRn.

In certain embodiments, the physiological pH is about 7.4.

In certain embodiments, the present disclosure is directed to an assay system, comprising: a) a first chamber and a second chamber, wherein each chamber comprises aqueous solution at physiological pH; b) a cell layer separating the first and second chamber, wherein the cell layer can mediate recycling of a molecule from the first chamber, into the cell layer, and back into the first chamber; c) a detector for detecting the presence of a molecule in the first chamber; wherein the assay system is configured to determine the recycling of a plurality of molecules, wherein the determining comprises: introducing the plurality of molecules into the first chamber; incubating the plurality of molecules in the first chamber; replacing the aqueous solution in both the first and second chambers; and measuring the amount of the plurality of molecules that is released from the cell layer into the first chamber.

In certain embodiments, the assay system further comprises a detector for detecting the presence of a molecule in the second chamber; the cell layer can mediate the transcytosis of a molecule from the first chamber to the second chamber; and wherein the assay system is configured to determine the transcytosis of a plurality of molecules across the cell layer, wherein determining transcytosis comprises, after the aqueous solution has been replaced, measuring the amount of the plurality of molecules in the second chamber.

In certain embodiments, the present disclosure is directed to a kit comprising the assay systems and/or methods of the present disclosure.

### BRIEF DESCRIPTION OF THE DRAWINGS

FIG. 1 depicts a schematic of a mechanism of action of the FcRn that involves a pH-dependent capture and release, and intracellular trafficking pathways comprising recycling, transcytosis, and lysosomal degradation.

FIG. 2 depicts a schematic of a particular embodiment of the recycling assays of the present disclosure.

FIG. 3A depicts a schematic of a sensitive human IgG-specific enzyme-linked immunosorbent assay (ELISA) used to quantify recycling as described in the Examples.

FIG. 3B provides a standard curve of the ELISA assay depicted in FIG. 3A, demonstrating the lower limit of detection.

FIG. 4 shows that recycling and transcytosis output of various humanized IgG1 antibodies with differential Fc-FcRn binding affinity are impacted by human FcRn.

FIGS. 5A-5B provides a comparison of time-dependent recycling measurements demonstrating similar recycling kinetics in four therapeutic antibodies. FIG. 5A provides the non-normalized data, and FIG. 5B provides normalized data.

FIG. 6 shows the transcytosis/recycling (T/R) ratio for a wildtype (WT) BACE-1 antibody molecule and several sequence variants.

FIG. 7 shows the relationship between transcytosis/recycling (T/R) ratio and the brain serum ratio in cynomolgus monkeys for five anti-BACE1 mAb variants.

FIGS. 8A-8D provide clearance rate in humans compared to calculated pI (FIG. 8A), Fv charge (FIG. 8B), HI sum (FIG. 8C), and the output of normalized recycling assays performed according to the methods and assays described herein, of a set of therapeutic antibodies. In FIGS. 8A-8C, there is low or no correlation between the clearance rate and the compared parameter. FIG. 8D demonstrates the correlation of clearance rate and normalized recycling output.

### DETAILED DESCRIPTION

In practicing the presently disclosed subject matter, many conventional techniques in molecular biology, microbiology, cell biology, biochemistry and immunology are used. These techniques are described in greater detail in, for example, Molecular Cloning: a Laboratory Manual 3rd edition, J. F. Sambrook and D. W. Russell, ed. Cold Spring Harbor Laboratory Press 2001; Recombinant Antibodies for Immunotherapy, Melvyn Little, ed. Cambridge University Press 2009; “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., ed., 1994); “A Practical Guide to Molecular Cloning” (Perbal Bernard V., 1988); “Phage Display: A Laboratory Manual” (Barbas et al., 2001). The contents of these references and other references containing standard protocols, widely known to and relied upon by those of skill in the art, including manufacturers' instructions are hereby incorporated by reference as part of the present disclosure.

### 1. Definitions

Unless otherwise defined, all terms of art, notations and other scientific terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art.

The terms “comprise(s),” “include(s),” “having,” “has,” “can,” “contain(s),” and variants thereof, as used herein, are intended to be open-ended transitional phrases, terms or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean, in certain embodiments, within 3 or more than 3 standard deviations, per the practice in the art. Alternatively, “about” can mean, in certain embodiments, a range of up to 20%, up to 10%, up to 5%, or of up to 1% of a given value. Alternatively, particularly with respect to biological systems or processes, the term can mean within an order of magnitude, e.g., within 5-fold, or within 2-fold, of a value.

As used herein, the terms “medium” and “cell culture medium” refer to a nutrient source used for growing or maintaining cells. As is understood by a person of skill in the art, the nutrient source may contain components required by the cell for growth and/or survival or may contain components that aid in cell growth and/or survival. Vitamins, essential or non-essential amino acids (e.g., cysteine and cystine), and trace elements (e.g., copper) are examples of medium components. Any media provided herein may also be supplemented with any one or more of insulin, plant hydrolysates and animal hydrolysates.

As used herein, the phrase “pharmacokinetic (PK) parameter” refers to any of a variety of PK parameters known in the art, including, but not limited to, in vivo clearance (CL), volume of distribution (Vd), area under the curve (AUC), bioavailability, maximum/minimum plasma concentrations (Cmax/Cmin), or in vivo half-life (t½) of the plurality of molecules.

As used herein, the term “clearance” refers to the rate at which a molecule or a polypeptide is removed from the bloodstream of an animal. The animal may be, for example, a mammal, such as a rodent (mouse, rat, hamster, guinea pig, or other rodent), non-human primate (such as cynomolgus monkey), dog, or human.

As used herein, the term “physiological pH” refers to a pH of about 6.5 to about 8.0. In certain embodiments the physiological pH value is any value between about 6.5 and about 8.0, e.g., about 6.6, about 6.7, about 6.8, about 6.9, about 7.0, about 7.1, about 7.2, about 7.3, about 7.4, about 7.5, about 7.6, about 7.7, about 7.8, or about 7.9, or any range within the range of about 6.5 to about 8.0.

“Culturing” a cell refers to contacting a cell with a cell culture medium under conditions suitable to the survival and/or growth and/or proliferation of the cell.

As used herein, a “molecule” refers generally to molecule that is the subject of the assay. For example, but not by way of limitation, the molecule can be any molecule that naturally binds to FcRn (e.g., polypeptides, antibodies, Fc-containing molecules, etc.) or has been engineered to bind to FcRn, such as, but not limited to a an albumin-containing molecule, a molecule engineered to bind to FcRn via peptide tags or other amino acid sequences, an antibody, an antibody fragment, or a polyclonal or monoclonal antibody as defined below. Such polypeptides, proteins, antibodies, antibody fragments, or polyclonal or monoclonal antibodies can include non-naturally occurring aspects, including, but not limited to, non-naturally occurring amino acids, non-amino acid linkers or spacers, and can include conjugates to other compositions, e.g., small molecule or large molecule therapeutics.

As used herein, “polypeptide” refers generally to peptides and proteins having more than about ten amino acids. The polypeptides may be homologous to the host cell or may be exogenous, meaning that they are heterologous, i.e., foreign, to the host cell being utilized, such as a human protein produced by a Chinese hamster ovary cell, or a yeast polypeptide produced by a mammalian cell. In certain embodiments, mammalian polypeptides (polypeptides that were originally derived from a mammalian organism) are used and, in certain embodiments, the polypeptides of the present disclosure are directly secreted into the medium.

The term “protein” is meant to refer to a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15-20 kD, and in certain embodiments, at least about 20 kD. Examples of proteins encompassed within the definition herein include all mammalian proteins, in particular, therapeutic and diagnostic proteins, such as therapeutic and diagnostic antibodies, and, in general proteins that contain one or more disulfide bonds, including multi-chain polypeptides comprising one or more inter- and/or intrachain disulfide bonds.

The term “antibody” is used herein in the broadest sense and encompasses various types of antibodies and antibody structures, including, but not limited to, polyclonal or monoclonal antibodies, human, humanized or chimeric antibodies, multispecific antibodies, e.g., bispecific antibodies, and antibody fragments that exhibit the desired antigen-binding activity.

An “antibody fragment,” “antigen-binding portion” of an antibody (or simply “antibody portion”) or “antigen-binding fragment” of an antibody, as used herein, refers to a molecule other than an intact antibody that comprises a portion of an intact antibody that binds the antigen and/or epitope to which the intact antibody binds. Examples of antibody fragments include but are not limited to Fv, Fab, Fab′, Fab′-SH, F(ab′)2; diabodies; linear antibodies; single-chain antibody molecules (e.g., scFv); and antibody fragments formed from multispecific, e.g., bispecific antibodies.

The term “monoclonal antibody” or “mAb” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “hybridoma” refers to a hybrid cell line produced by the fusion of an immortal cell line of immunologic origin and an antibody producing cell. The term encompasses progeny of heterohybrid myeloma fusions, which are the result of a fusion with human cells and a murine myeloma cell line subsequently fused with a plasma cell, commonly known as a trioma cell line. Furthermore, the term is meant to include any immortalized hybrid cell line which produces antibodies such as, for example, quadromas. See, e.g., Milstein et al., Nature, 537:3053 (1983).

As used herein, the term “cell,” refers to animal cells, mammalian cells, cultured cells, host cells, recombinant cells, and recombinant host cells. Such cells are generally cell lines obtained or derived from mammalian tissues which are able to grow and survive when placed in media containing appropriate nutrients and/or growth factors.

As used herein, the term “heterologous gene” refers to a gene encoding a protein that is foreign to the host cell being utilized, such as gene encoding a human protein produced by in Chinese hamster ovary cell, or a gene encoding a yeast polypeptide produced in a mammalian cell.

“Plurality of molecules”, as used herein, includes a plurality of a single molecule, and a plurality of distinct molecules. A plurality of a single molecule, for example, may be two or more physical molecules of the same antibody, two or more physical molecules of the same antibody fragment, two or more physical molecules of the same polypeptide, or two or more physical molecules of the same Fc-containing molecule, or two or more physical molecules of another molecule of interest. A plurality of distinct molecules may include mixtures of one molecule type (e.g., physical molecules of at least two different antibodies, physical molecules of at least two different antibody fragments, physical molecules of at least two different polypeptides, or physical molecules of at least two Fc-containing molecules), but may also include mixtures between molecule types (e.g., physical molecules of one or more antibodies and one or more polypeptides; or one or more antibody fragments, one or more polypeptides, and one or more Fc-containing molecules, etc.).

“Test molecule” and “molecule of interest” as used herein refers to the identity of the plurality of molecules being evaluated in the assays, methods, kits, and systems being described herein. As described for plurality of molecules, such terms encompass both a single type of molecule (e.g., one antibody, antibody fragment, polypeptide, or Fc-containing molecule), and two or more types of molecules (e.g., two or more antibodies, antibody fragments, polypeptides, or Fc-containing molecules, or a combination of said categories).

“Small molecule” as used herein refers to a molecule, other than a protein or polypeptide, with a molecular weight of about 2000 daltons or less, or preferably about 500 daltons or less.

### 2. Overview

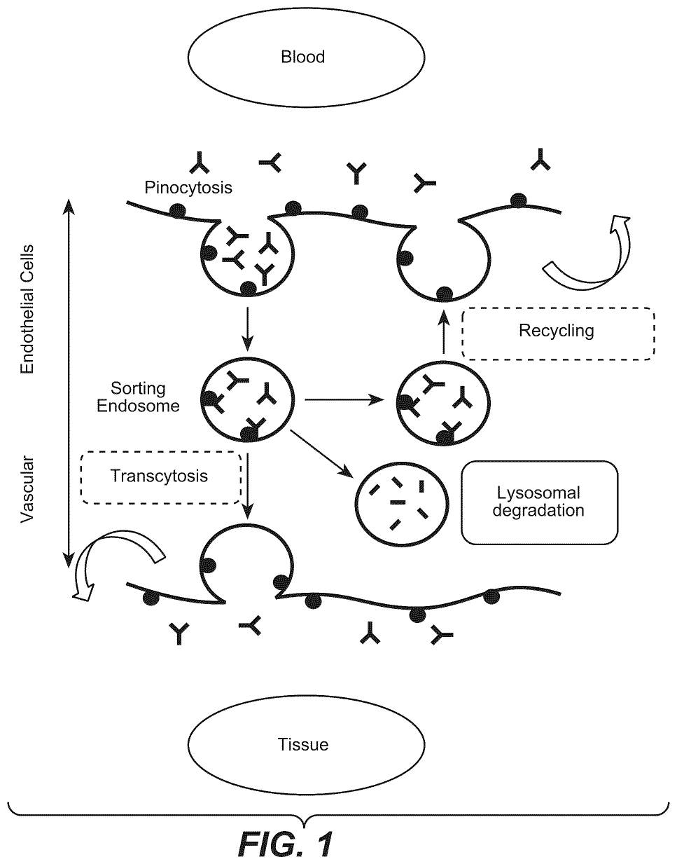

The present disclosure relates to methods, assays, assay systems, kits and compositions useful for measuring the movement or transportation of a molecule of interest to and from a cellular membrane, such as the recycling of a molecule of interest (as depicted in FIG. 1). The present disclosure further relates to methods assays, assay systems, kits, and compositions useful for measuring the transcytosis and recycling of a molecule of interest (as depicted in FIG. 1). In particular, the present disclosure relates to in vitro receptor-dependent recycling and transcytosis assays for evaluating the PK parameters such as clearance rates and/or half-lives of therapeutic antibody molecules. As disclosed in detail herein, the recycling, optionally in further combination with transcytosis, of a molecule of interest (e.g., antibody, antibody fragment, polypeptide, or Fc-containing molecule) measured by the methods of the instant disclosure may be highly associated with PK properties of a cell, such as the in vivo clearance of that molecule. Thus, the claimed methods for measuring the recycling, and in some instances the combination of transcytosis and recycling, of a molecule of interest can be used to evaluate the in vivo clearance of the molecule and other PK parameters such as half-life, volume of distribution (Vd), area under the curve (AUC), bioavailability, and maximum/minimum plasma concentrations (Cmax/Cmin), of the plurality of molecules. Still further, the recycling, optionally in further combination with transcytosis, of a molecule of interest (e.g. an antibody or antibody fragment thereof), measured by the methods of the instant disclosure may be highly associated with tissue penetrance of said molecule of interest in vivo. Thus, in some embodiments, the claimed methods for measuring the recycling, and in some instances the combination of transcytosis and recycling, of a molecule of interest can be used to evaluate the in vivo tissue penetrance of said molecule of interest. Tissue penetrance may include, for example, brain penetrance.

In certain embodiments, the methods include measuring the transcellular recycling of a plurality of the same molecule of interest from a first chamber to same chamber by measuring the uptake of the molecule or molecules from a solution in a first chamber and the return of those molecule to the first chamber after the original solution has been replaced by a second solution, wherein each of the first and second chambers have a physiological pH value (e.g., pH=about 7.4). In some embodiments, the transcellular transportation of a plurality of the same molecule of interest from a first chamber to a second chamber is also evaluated. In other embodiments, the methods include measuring transcellular recycling of a plurality of different molecules of interest (e.g., two, three, four, five, or more types of molecules of interest), which optionally may include evaluating the transcellular transportation of a plurality of different molecules of interest from a first chamber to a second chamber. The employment of said assays is performed using a cell layer separating the first and second chambers. In certain embodiments, the cell layer stably expresses one or more receptors that mediate molecular transport. Such receptor may be any receptor of interest, including but not limited to a transferrin receptor, an Fc receptor, megalin, or cubulin.

Thus, for example, in one aspect, a cell-based assay employing MDCK cells stably expressing human FcRn and β2-microglobulin genes is provided herein to measure the recycling efficiency, and further optionally transcytosis, of polypeptides, antibodies, or other FcRn-binding molecules under conditions relevant to the FcRn-mediated IgG salvage pathway. This transcytosis and recycling assay is conducted under physiological pH condition and is designed to assess outcome from combined effects of two or more of non-specific binding of IgG to cells, its uptake via pinocytosis, its pH-dependent interactions with FcRn, and dynamics of intracellular trafficking and sorting processes. In this particular embodiment, the expression of FcRn is required to promote the transcytosis and recycling of test molecules in the assay and contributes directly to the observed correlation. Further, assays such as provided herein were able to correctly rank order clearance rates of charge or glycosylation variants of Fc-containing molecules in preclinical species. The results provided in the Examples support the utility of the assays disclosed herein as cost effective and animal-saving screening tools for evaluation of polypeptides, antibodies, or other FcRn-binding molecules , including antibody drug candidates during lead selection and optimization, and process development for desired pharmacokinetic properties.

The present disclosure also relates to methods and compositions useful for determining the tissue penetrance of a plurality of molecules by determining the transcytosis/recycling (T/R) ratio of the plurality of molecules. In certain embodiments, the methods include determining the brain penetrance of the plurality of molecules based on the ratio of transcytosed to recycled molecules. The claimed methods for measuring the transcytosis and recycling and determining the T/R ratio of a molecule of interest can be used to evaluate brain penetrance of the molecule and other PK parameters such as half-life. The in vitro assays such as provided herein demonstrated correlation with brain penetrance of Fc-containing molecules in preclinical species. Thus, the results provided in the Examples support the utility of the assays disclosed herein as cost effective and animal-saving screening tools for evaluation of polypeptides, antibodies, or other FcRn-binding molecules, including antibody drug candidates during lead selection and optimization, and process development for desired tissue penetrance properties.

The present disclosure relates to methods, assays, assay systems, kits and compositions provided herein may be used to evaluate the movement or transportation (e.g., recycling, or recycling and transcytosis), one or more PK parameters, and/or tissue penetrance (e.g., brain penetrance) of a plurality of the same molecule of interest (e.g., the same type of antibody, antibody fragment, polypeptide, or Fc-containing molecule), or a plurality of two or more molecules of interest (e.g., two or more molecules of interest selected from antibodies, antibody fragments, polypeptides, Fc-containing molecules, or any mixtures thereof).

### 3. Recycling, and Recycling and Transcytosis Assays

Recycling refers to the vesicular transport of macromolecules from one side of a cell to the same side. Recycling is a mechanism for transcellular transport in which a cell encloses extracellular material in an invagination of the cell membrane to form a vesicle, then moves the vesicle within the cell to eject the material through the same cell membrane by the reverse process. See, FIG. 1.

Transcytosis, which refers to the vesicular transport of macromolecules from one side of a cell to the other, is a strategy used by multicellular organisms to selectively move material between two environments without altering the unique compositions of those environments. Transcytosis is a mechanism for transcellular transport in which a cell encloses extracellular material in an invagination of the cell membrane to form a vesicle, then moves the vesicle across the cell to eject the material through the opposite cell membrane by the reverse process. See, FIG. 1.

In a typical FcRn-mediated transcytosis assay, FcRn-expressing MDCK cells are grown to confluency on filter membranes in trans-well plates, and before the assay, a pH gradient is created by filing the inner chamber with acidic assay buffer (pH<6.0) and the outer chamber with basic assay buffer (pH>7.4). This assay design takes advantage of the pH-dependent binding characteristic of FcRn to facilitate the cellular uptake of test molecules via binding with the FcRn on cell surface under acidic pH and the release of the test molecules under basic pH, both of which improve the robustness and sensitivity of the assay. However, the predictive assessment of PK behavior of test molecules using said assay could be improved. Since the transfected cells typically express high level of FcRn on cell surface and the test antibodies are incubated with the cells in an acidic environment, it is believed that test molecules (such as antibodies) exhibiting high binding affinity toward FcRn at acidic pH readily bind to FcRn and enter the cells via FcRn-mediated endocytosis. This is in contrast to what typically happens in vivo, where polypeptides or antibodies bind minimally to cell surface FcRn under physiologic pH, and cellular uptake is mainly mediated by non-specific fluid-phase pinocytosis. As a consequence, without wishing to be bound by theory, it is believed that the output of the pH gradient transcytosis assay can be heavily influenced by the polypeptide's or antibody's FcRn binding affinity at acidic pH and therefore may not always reflect adequately the contribution of other factors that impact PK, such as electrostatic interactions and intracellular trafficking parameters. In addition, the artificial pH conditions may be intrinsically detrimental to cells, limiting the duration of the assay and potentially creating additional assay artifacts.

Accordingly, and as described in greater detail in the Examples, provided herein is an improved method for evaluating the transport of molecules within a cellular environment, which may further be used to more accurately determine or measure one or more PK parameters (e.g., in vivo clearance (CL), volume of distribution (Vd), area under the curve (AUC), bioavailability, maximum/minimum plasma concentrations (Cmax/Cmin), or in vivo half-life (t½)), or tissue penetrance (such as brain penetrance). Thus, for example, in certain embodiments, the present disclosure provides methods for determining or measuring the recycling of a plurality of molecules. For example, the determining or measuring can comprise introducing the plurality of molecules (including, e.g., antibodies, antibody fragments, polypeptides, or Fc-containing molecules) into a first chamber, wherein: the first chamber is separated from a second chamber by a cell layer, and the first and second chambers comprise an aqueous solution; incubating the plurality of molecules in the first chamber; replacing the aqueous solution in both the first and second chambers; and measuring the amount of the plurality of molecules that is released from the cell layer into the first chamber; wherein the aqueous solution is at physiological pH, and the cell layer comprises cells that express a receptor that mediates molecular transport.

In certain embodiments of the methods, assays, and systems provided herein, the physiological pH value is about 6.5 to about 8.0. In certain embodiments the physiological pH value is any value between about 6.5 and about 8.0, e.g., about 6.6, about 6.7, about 6.8, about 6.9, about 7.0, about 7.1, about 7.2, about 7.3, about 7.4, about 7.5, about 7.6, about 7.7, about 7.8, or about 7.9, or any range within the range of about 6.5 to about 8.0. In certain embodiments, the pH of each chamber can differ, as long as both chambers have a physiological pH. For example, but not by way of limitation, the first chamber can have a pH of 6.5 while the second chamber can have a pH of 8.0, or vice versa. In certain embodiments, the physiological pH can be about 7.4.

In certain embodiments, the present disclosure is directed to a method of determining a pharmacokinetic (PK) parameter of a plurality of molecules (including, e.g., antibodies, antibody fragments, polypeptides, or Fc-containing molecules), comprising: a) introducing the plurality of molecules into a first chamber, wherein the first chamber is separated from a second chamber by a cell layer, and the first and second chambers comprise an aqueous solution; wherein the aqueous solution is at physiological pH, and the cell layer comprises cells that express a receptor that mediates molecular transport; b) measuring the amount of the plurality of molecules that is recycled from the first chamber into the cell layer and back to the first chamber; and c) determining the PK parameter based on the amount of the plurality of molecules that is recycled. In certain embodiments, measuring the plurality of molecules (including, e.g., antibodies, antibody fragments, polypeptides, or Fc-containing molecules) that is recycled comprises: after introducing the plurality of molecules into the first chamber, incubating the plurality of molecules in the first chamber; replacing the aqueous solution in both the first and second chambers; and measuring the amount of the plurality of molecules that is released from the cell layer into the first chamber. In certain embodiments, the plurality of molecules (including, e.g., antibodies, antibody fragments, polypeptides, or Fc-containing molecules) is incubated in the first chamber from about 1 hour to about 48 hours, about 1 hour to about 30 hours, about 1 hour to about 24 hours, about 1 hour to about 12 hours, about 1 hour to about 6 hours, or about 1 hour to about 4 hours, or any values therein, such as about 1 hour, about 2 hours, about 4 hours, about 6 hours, about 8 hours, about 12 hours, or about 18 hours. In certain embodiments, replacing the aqueous solution comprises washing the first chamber. In certain embodiments, the method further comprises incubating the first and second chambers with the replacement aqueous solution prior to measuring. In certain embodiments, the first and second chambers are incubated with the replacement aqueous solution for between about 10 minutes and about 12 hours, between about 20 minutes to about 12 hours, between about 30 minutes to about 6 hours, between about 20 minutes to about 5 hours, or between about 1 hour to about 4 hours, or any values therein, such as 30 minutes, 1 hour, 2 hours, 3 hours, or 4 hours, prior to measuring. In certain embodiments, the incubation is at a temperature of between about 35° C. to about 39° C., such as about 37° C. In some embodiments, a quantification method with a lower level lower limit of quantification (LLOQ) of at least 200 pg/mL, at least 100 pg/mL, at least 50 pg/mL, or signle-digit pg/mL is used. Such methods may include, for example, ELISA using a high-affinity capture reagent, which may be, for example a biotinylated mouse anti-human IgG-Fc, such as biotin-R10Z. In certain embodiments, the plurality of molecules is a plurality of the same molecule (e.g., the same type of antibody, antibody fragment, polypeptide, or Fc-containing molecule), while in other embodiments the plurality of molecules is a plurality of a mixture of molecules (e.g., one or more different antibodies, antibody fragments, polypeptides, or Fc-containing molecules, or a mixture thereof).

In certain embodiments, the measure of recycling can be determined by measuring the uptake of the molecule or molecules (including, e.g., antibodies, antibody fragments, polypeptides, or Fc-containing molecules) from a solution in a first chamber and the return of those molecule to the first chamber after the original solution has been replaced by a second solution. In some embodiments, wherein transcytosis is also evaluated, the measure of transcytosis can be determined by measuring the transcytosis of the molecule or molecules (including, e.g., antibodies, antibody fragments, polypeptides, or Fc-containing molecules) from a solution in a first chamber to a solution in a second chamber, wherein the first and second chamber are separated by a cell monolayer and the solution in each chamber is at physiological pH. In certain embodiments, the cell or plurality of cells is eukaryotic, such as mammalian, for example derived from a rodent (such as mouse, rate, guinea pig, hamster, or other mammal), dog, non-human primate (such as cynomolgus monkey), or human. Cells that may be used are described in greater detail herein. In certain embodiments, the cell or a plurality of cells can be Madin-Darby Canine Kidney (MDCK) cells. In some embodiments, the cell or plurality of cells are wild type. In other embodiments, they express one or more heterologous receptors of interest. In certain embodiments, the cells comprise at least one heterologous gene. In certain embodiments, the at least one heterologous gene can be selected from the group consisting of a FCGRT gene and a B2M gene. In certain embodiments, the cells can express a heterologous cell surface protein. In certain embodiments, the heterologous cell surface protein can comprise a neonatal Fc receptor (FcRn). In certain embodiments, measuring the recycling and optionally the transcytosis of the molecule comprises the use of an enzyme-linked immunosorbent assay (ELISA), liquid-scintillation counting (LSC), quantitative PCR, or a fluorescence reader system. In some embodiments, measuring the recycling and optionally the transcytosis of the molecule comprises the use of a quantification method capable of evaluating sub-nanomolar concentrations of the molecule of interest. In some embodiments, a quantification method with a lower level lower limit of quantification (LLOQ) of at least 200 pg/mL, at least 100 pg/mL, or at least 50 pg/mL is used. Such methods may include, for example, ELISA using a high-affinity capture reagent, which may be, for example a biotinylated mouse anti-human IgG-Fc, such as biotin-R10Z. Such sensitive ELISA methods are described in the Examples section of the present disclosure. In some embodiments, such a sensitive quantification method is used to measure recycling, but a less sensitive method is used to evaluate transcytosis (e.g. with an LLOQ at nanomolar concentrations). In some embodiments, a sensitive automated immunoassay platform is used to quantify recycled and/or transcytosed molecules (e.g. from Quanterix, Gyrolab, or Singulex). In certain embodiments, the methods of the present disclosure can further comprise determining the in vivo clearance (CL), volume of distribution (Vd), area under the curve (AUC), bioavailability, maximum/minimum plasma concentrations (Cmax/Cmin), or in vivo half-life (t½) of the plurality of molecules based on the quantity of molecules measured. In certain embodiments, the molecule can be a molecule that naturally binds to a receptor of interest, or a molecule engineered to bind to a receptor of interest, wherein the receptor of interest is expressed by the cell or plurality of cells. Thus, in some embodiments, the molecule is a molecule that naturally binds to FcRn or a molecule engineered to bind to FcRn. In certain embodiments, the molecule can be an Fc-containing molecule. In certain embodiments, the molecule can be an antibody. In certain embodiments, the antibody can be a monoclonal antibody. In certain embodiments, the antibody can be an anti-IgE antibody, an anti-VEGF antibody, an anti-integrin antibody (e.g., anti-a4137 integrin antibody), anti-IL-6 antibody, anti-TNFa antibody, anti-BACE1 antibody, or anti-gD antibody. In some embodiments, the antibody can be omalizumab, bevacizumab, vedolizumab, tocilizumab, adalimumab, anti-BACE1 WT, anti-BACE1 variant YEY (mutations: M252Y; N286E; N434Y), anti-BACE1 variant YQAY (mutations: M252Y; T307Q; Q311A; N434Y), anti-BACE1 variant YPY (mutations: M252Y; V308P; N434Y), anti-BACE1 variant YY (mutations: M252Y; N434Y), anti-BACE1 variant YLY1 (Q6) (mutations: M252Y; M428L; N434Y; Y436I), or anti-BACE1 variant YIY (T3) (mutations: M252Y; Q 3111; N434Y). In certain embodiments, the molecule can be an albumin-containing molecule. In certain embodiments the physiological pH value is about 6.5 to about 8.0. In certain embodiments the physiological pH value is any value between about 6.5 and about 8.0, e.g., about 6.6, about 6.7, about 6.8, about 6.9, about 7.0, about 7.1, about 7.2, about 7.3, about 7.4, about 7.5, about 7.6, about 7.7, about 7.8, or about 7.9, or any range within the range of about 6.5 to about 8.0. In certain embodiments, the pH of each chamber can differ, as long as both chambers have a physiological pH. For example, but not by way of limitation, the first chamber can have a pH of 6.5 while the second chamber can have a pH of 8.0, or vice versa. In certain embodiments, the physiological pH can be about 7.4. In certain embodiments, a PK parameter is determined, such as a measure of in vivo clearance (CL), volume of distribution (Vd), area under the curve (AUC), bioavailability, maximum/minimum plasma concentrations (Cmax/Cmin), or in vivo half-life (t½) of the plurality of molecules. In other embodiments, separately or in combination, tissue penetrance is determined, such as brain penetrance.

In certain embodiments, the present disclosure is directed to an assay system, comprising: a) a first chamber and a second chamber, wherein each chamber comprises aqueous solution at physiological pH; b) a cell layer separating the first and second chamber, wherein the cell layer can mediate recycling of a molecule from the first chamber, into the cell layer, and back into the first chamber; c) a detector for detecting the presence of a molecule in the first chamber; wherein the assay system is configured to determine the recycling of a plurality of molecules, wherein the determining comprises: introducing the plurality of molecules (including, e.g., antibodies, antibody fragments, polypeptides, or Fc-containing molecules) into the first chamber; incubating the plurality of molecules in the first chamber; replacing the aqueous solution in both the first and second chambers; and measuring the amount of the plurality of molecules that is released from the cell layer into the first chamber. The cells forming the cell layer may be any that can be stably grown to form cell layer, as described further herein. In certain embodiments the cells can be seeded at a density of about 1×10⁵ cells/well in cell growth medium. The cell growth medium may be any suitable medium for growing the selected cells. In certain embodiments the cell growth medium can be DMEM High Glucose supplemented with 10% FBS, 100 units of Penicillin/Streptomycin, and 5 μg/mL of Puromycin. In some embodiments, the volumes of medium in the inner and outer chambers are independently from 25 μL to 500 μL, or 50 μL to 350 μL , or 50 μL to 200 μL. In certain embodiments the medium in the inner chamber can be 100 μL and the medium in the outer chamber can be 200 μL. In certain embodiments, the cells can be used on the second day post-plating. In certain embodiments, the test molecules can be added to a final concentration of about 100 μg/mL (0.67 μM) and incubated for 24 hours in a 37° C. tissue culture incubator. In certain embodiments, Lucifer Yellow (Lucifer Yellow CH, dilithium salt; Sigma Aldrich; St. Louis MO) can be prepared in the cell growth medium and can be added to the final 90 minutes of the 24-hour assay incubation. In certain embodiments, the level of passive passage of Lucifer Yellow during the assay can be calculated by dividing the florescent signal in samples from the outer chamber by that of the inner chamber. In certain embodiments, transcytosis and recycling results from wells exhibiting greater than 0.1% of passive passage of Lucifer Yellow in the outer chamber can be discarded, that is, only wells wherein less than 0.1% of passive passage of Lucifer Yellow are used. In some embodiments, the detector system is one that is capable of evaluating sub-nanomolar concentrations of the molecule of interest (including, e.g., antibodies, antibody fragments, polypeptides, or Fc-containing molecules), such as with an LLOQ of at least 200 pg/mL, at least 100 pg/mL, or at least 50 pg/mL. In some embodiments, the detector system comprises an ELISA assay using a high-affinity capture reagent, which may be, for example a biotinylated mouse anti-human IgG-Fc, such as biotin-R10Z. Such sensitive methods are described herein in the Examples.

In certain embodiments, the test molecule (including, e.g., antibodies, antibody fragments, polypeptides, or Fc-containing molecules) can be added into the outer or the inner chamber of a trans-well assay and recycling can be detected by measuring the amount of test molecule present in the same chamber (e.g., the outer or inner chamber, respectively), following an appropriate incubation period (pulse step), removing the medium, and replacing it (chase step). In some embodiments, the transcytosis of a test molecule is also detected, by measuring the amount of test molecule present in the opposite chamber (e.g., the inner or outer chamber, respectively). In certain embodiments the test molecule is recycled from a chamber exposed to the apical membrane of a cell back to the same chamber. In some embodiments, the test molecule is recycled from a chamber exposed to the basolateral membrane of a cell back to the same chamber. In still further embodiments, the test molecule is transcytosed from a chamber exposed to the apical membrane of a cell to a chamber exposed to the basolateral membrane of a cell.

In certain embodiments, the present disclosure provides methods for determining or predicting a PK parameter of one or more molecules (including, e.g., antibodies, antibody fragments, polypeptides, or Fc-containing molecules), comprising: a) introducing the molecule(s) into a first chamber of two chambers, where the first chamber is separated from the second chamber by a cell or plurality of cells and wherein each of the first and second chambers has a physiological pH value; and b) measuring the number of molecules recycled from the first chamber to the first chamber. In certain embodiments the physiological pH value is about 6.5 to about 8.0. In certain embodiments the physiological pH value is any value between about 6.5 and about 8.0, e.g., about 6.6, about 6.7, about 6.8, about 6.9, about 7.0, about 7.1, about 7.2, about 7.3, about 7.4, about 7.5, about 7.6, about 7.7, about 7.8, or about 7.9, or any range within the range of about 6.5 to about 8.0. In certain embodiments, the pH of each chamber can differ, as long as both chambers have a physiological pH. For example, but not by way of limitation, the first chamber can have a pH of 6.5 while the second chamber can have a pH of 8.0, or vice versa. In certain embodiments, the physiological pH can be about 7.4. In some embodiments, the PK parameter is in vivo clearance (CL), volume of distribution (Vd), area under the curve (AUC), bioavailability, maximum/minimum plasma concentrations (Cmax/Cmin), or in vivo half-life (t½). In certain embodiments, the PK parameter is in vivo clearance.

In certain embodiments, the assay system further comprises a detector for detecting the presence of a molecule in the first chamber; the cell layer can mediate the recycling of a molecule from the first chamber to the first chamber; and wherein the assay system is configured to determine the recycling of a plurality of molecules from the cell layer back into the first chamber, wherein determining recycling comprises, after the aqueous solution has been replaced, measuring the amount of the plurality of molecules in the first chamber. In some embodiments, transcytosis is also measured. In certain embodiments, the assay system further comprises a detector for detecting the presence of a molecule in the second chamber; the cell layer can mediate the transcytosis of a molecule from the first chamber to the second chamber; and wherein the assay system is configured to determine the transcytosis of a plurality of molecules across the cell layer, wherein determining transcytosis comprises, after the aqueous solution has been replaced, measuring the amount of the plurality of molecules in the second chamber. In some embodiments, the detector for detecting the presence of a molecule in the first chamber is capable of evaluating sub-nanomolar concentrations of the molecule or molecules of interest (including, e.g., antibodies, antibody fragments, polypeptides, or Fc-containing molecules), such as with an LLOQ of at least 200 pg/mL, at least 100 pg/mL, or at least 50 pg/mL. In some embodiments, the detector for detecting the presence of a molecule in the second chamber has similar sensitivity. In other embodiments, it does not, for example, it may have an LLOQ in the nanomolar concentration range

In certain embodiments, the present disclosure provides assays for measuring the transcellular transportation of a plurality of a the same molecule or a plurality of distinct molecules, comprising a first chamber and a second chamber, each of the first and second chambers having a physiological pH value, wherein the first chamber receives a plurality of the molecules and is configured to allow for transcytosis of the molecules to the second chamber and for recycling of the molecules back into the first chamber. In certain embodiments the physiological pH value is about 6.5 to about 8.0. In certain embodiments the physiological pH value is any value between about 6.5 and about 8.0, e.g., about 6.6, about 6.7, about 6.8, about 6.9, about 7.0, about 7.1, about 7.2, about 7.3, about 7.4, about 7.5, about 7.6, about 7.7, about 7.8, or about 7.9, or any range within the range of about 6.5 to about 8.0. In certain embodiments, the pH of each chamber can differ, as long as both chambers have a physiological pH. For example, but not by way of limitation, the first chamber can have a pH of 6.5 while the second chamber can have a pH of 8.0, or vice versa. In certain embodiments, the physiological pH can be about 7.4.

In certain embodiments, the present disclosure provides assays for determining or predicting one or more PK parameters of one or more molecules, comprising a first chamber and a second chamber, each of the first and second chambers having a physiological pH value, wherein the first chamber receives a plurality of the molecules and is configured to allow for recycling of the molecules from the first chamber back into the first chamber, and optionally is configured to allow for transcytosis of the molecules from the first chamber to the second chamber. In certain embodiments the physiological pH value is about 7.4. In some embodiments, the PK parameter is in vivo clearance (CL), volume of distribution (Vd), area under the curve (AUC), bioavailability, maximum/minimum plasma concentrations (Cmax/Cmin), or in vivo half-life (t½). In some embodiments, the PK parameter is in vivo clearance.

In certain embodiments, the first chamber can comprise a monolayer of cells. In certain embodiments, the cells employed in the assay are selected from the listing provided in Section 4 entitled ‘Cells’, below. In certain embodiments, the cells employed in the assay are eukaryotic cells. In some embodiments, they are mammalian cells. In certain embodiments, they are kidney cells. In some embodiments, the kidney cells are rodent, non-human primate, human, or canine. In some embodiments, the cells are MDCK cells. In certain embodiments, the cells used in the methods described in the present disclosure can comprise at least one heterologous gene. In certain embodiments, the at least one heterologous gene can be selected from the group consisting of a FCGRT gene and a B2M gene. In certain embodiments, the cells used in the methods described in the present disclosure can express a cell surface protein. In certain embodiments, the cell surface protein comprises a Fc receptor (FcR). In certain embodiments, the cell surface protein comprises a neonatal Fc receptor (FcRn).

In certain embodiments, the molecules can be labeled or unlabeled. Non-limiting examples of labeled molecules include 3H-labeled, fluorescently labeled molecules, or radioisotopes, e.g., 1-125 and P-32.

In certain embodiments, the methods of the present disclosure include measuring the number of the recycled molecules, and optionally transcellularly transported molecules. In certain, non-limiting embodiments, measuring the number of the recycled or transcellularly transported molecules can be performed by enzyme-linked immunosorbent assays (ELISA), liquid-scintillation counting (LSC), quantitative PCR, fluorescence reader systems, confocal microscopy, or live cell imaging systems, or any combinations thereof. The use of different methods of detection for recycled vs. transcytosed molecule is contemplated. In some embodiments, the method of measuring is one that is capable of evaluating sub-nanomolar concentrations of the molecule or molecules of interest (including, e.g., antibodies, antibody fragments, polypeptides, or Fc-containing molecules), such as with an LLOQ of at least 200 pg/mL, at least 100 pg/mL, or at least 50 pg/mL. In some embodiments, the detector system comprises an ELISA assay using a high-affinity capture reagent, which may be, for example a biotinylated mouse anti-human IgG-Fc, such as biotin-R10Z. In certain embodiments, different methods are used, wherein the method used to measure recycling is capable of evaluating sub-nanomolar concentrations, and the method used to measure transcytosis is not. Sensitive automated immunoassay platforms may be used to quantify recycled and/or transcytosed molecules (e.g. from Quanterix, Gyrolab, or Singulex).

In certain embodiments, the present disclosure provides FcRn-dependent cell-based assays for measuring recycling, and optionally transcytosis, of a molecule of interest (e.g., one or more antibodies, antibody fragments, polypeptides, or Fc-containing molecules) through MDCK cells expressing human FcRn and B2M under conditions resembling the FcRn-mediated IgG salvage pathway. In certain embodiments, the output of this assay involves not only Fc-FcRn interactions at physiological conditions, but also non-specific binding, cellular uptake, sorting and intracellular trafficking processes pertaining to in vivo PK behavior of the molecule of interest. In some embodiments, the molecule of interest is an IgG mAb, such as those described elsewhere herein. In some embodiments, the assay measures recycling, and optionally transcytosis, of two more molecules of interest.

In certain embodiments, the present disclosure provides methods and assays for correlating recycling with one or more PK properties in an animal of a molecule of interest (e.g., antibodies, antibody fragments, polypeptides, or Fc-containing molecules) with diverse structure, function and pharmacological properties. In certain embodiments, the PK property is in vivo clearance (CL), volume of distribution (Vd), area under the curve (AUC), bioavailability, maximum/minimum plasma concentrations (Cmax/Cmin), or in vivo half-life (t½). In some embodiments, the PK parameter is in vivo clearance. In some embodiments, the animal is a mammal, such as a human, or non-human primate such as cynomolgus monkey. In certain embodiments, the present disclosure provides methods and assays for correlating transcytosis and recycling with tissue penetrance in an animal of a molecule of interest (e.g., antibodies, antibody fragments, polypeptides, or Fc-containing molecules) with diverse structure, function and pharmacological properties. In some embodiments, the tissue penetrance is brain penetrance. In some embodiments, the animal is a mammal, such as a human, or non-human primate such as cynomolgus monkey.

In certain embodiments, the expression of FcRn may be required to promote the recycling, and optionally transcytosis (if also being evaluated), of a molecule of interest (including, e.g., antibodies, antibody fragments, polypeptides, or Fc-containing molecules), such as mAbs, in the assay and to contribute to the observed association between recycling and PK parameter, or tissue penetrance, being evaluated. In certain embodiments, methods and assays described in the present disclosure are able to rank order clearance rates of charge or glycosylation variants of Fc-containing molecules in preclinical species. In certain embodiments, the methods and assays described in the present disclosure can be used as a time efficient, cost effective and animal saving tool for evaluation of drug candidates (including, e.g., antibodies, antibody fragments, polypeptides, or Fc-containing molecules) during lead selection and optimization, and process development for desired pharmacokinetic properties.

In certain embodiments, the present disclosure provides cell-based assays and methods that measure recycling efficiency of drugs (including, e.g., antibodies, antibody fragments, polypeptides, or Fc-containing molecules) under physiologically relevant conditions. In certain embodiments, MDCK cells stably expressing human FcRn and β2-microglobulin genes are used in connection with the assays described herein.

In certain embodiments, the present disclosure provides methods and assays that are performed under physiological pH in normal cell culture medium supplemented with fetal bovine serum which supplies bovine albumin that is known to bind to human FcRn (Chaudhury C. et al., 2003, J. Exp. Med. 197, 315-322).

In certain embodiments, the present disclosure provides methods and assays wherein the molecules are taken up by cells via non-specific pinocytosis, interact with human FcRn in the presence of albumin and are recycled by the cells under conditions relevant to FcRn-mediated mechanism of action. In some embodiments, at least a portion of said molecules are recycled back to the side of the cell layer from which they were taken up. In some embodiments, at least a portion of said molecules are transcytosed to the opposite side of the cell layer. In certain embodiments, the present disclosure provides methods and assays for determining or predicting clearance of a molecule of interest (including, e.g., antibodies, antibody fragments, polypeptides, or Fc-containing molecules) in a mammal due to the method and/or assay's capability of assessing the combined effects of non-specific binding to cells, uptake via pinocytosis, pH-dependent interactions with FcRn, and/or dynamics of intracellular trafficking and sorting processes. In some embodiments, the mammal is a non-human primate, such as a cynomolgus monkey. In some embodiments, the mammal is a human.

In certain embodiments, the present disclosure provides methods and assays for determining or predicting tissue penetrance of a molecule of interest (including, e.g., antibodies, antibody fragments, polypeptides, or Fc-containing molecules). In certain embodiments, the tissue is a brain tissue. In certain embodiments, the present disclosure provides methods and assays for determining or predicting tissue penetrance of a molecule of interest by determining the T/R ratio of a molecule. In certain embodiments, the present disclosure provides methods and assays for determining or predicting tissue penetrance of a molecule of interest (including, e.g., antibodies, antibody fragments, polypeptides, or Fc-containing molecules) in a mammal due to the method and/or assay's capability of assessing the combined effects of non-specific binding to cells, uptake via pinocytosis, pH-dependent interactions with FcRn, and/or dynamics of intracellular trafficking and sorting processes and determining the T/R ratio of the molecule of interest. In some embodiments, the mammal is a non-human primate, such as a cynomolgus monkey. In some embodiments, the mammal is a human.

In certain embodiments, the present disclosure provides methods and assays used to correlate recycling, and further optionally the transcytosis, output obtained with the a PK parameter of a molecule of interest (including, e.g., antibodies, antibody fragments, polypeptides, or Fc-containing molecules) in a mammal. In some embodiments, the PK parameter is in vivo clearance (CL), volume of distribution (Vd), area under the curve (AUC), bioavailability, maximum/minimum plasma concentrations (Cmax/Cmin), or in vivo half-life (t½). In certain embodiments, the output of the methods and assays of the present disclosure can be the concentrations (ng/mL) of a recycled molecule in the medium of the inner chamber (e.g. first chamber), or optionally further of a transcytosed molecule in the medium of the outer chamber (e.g., second chamber), and the reportable value of the assay can be the average concentration of at least 2 replicate wells from the same plate. In certain embodiments, the output of the method and/or assay can be a measure of the rate of recycling or a measure of the relative recycling of the recycled molecule in reference to a control molecule. In certain embodiments, in addition to recycling, the output of the assay can be a measure of the rate of transcytosis, or a measure of the relative transcytosis of the transcytosed molecule in reference to a control molecule. In still further embodiments, the output of the assay can be a calculated value based on said recycling and optionally transcytosis, such as the ratio of recycling to transcytosis or ratio of transcytosis to recycling, alone or in reference to a control molecule. In some embodiments, the mammal is a non-human primate, such as a cynomolgus monkey. In some embodiments, the mammal is a human.

In yet further embodiments, the present disclosure provides methods and assays for use in determining the effect of one or more agents other than the test molecule on the recycling, transcytosis, tissue penetrance, PK parameters, or other characteristic of the test molecule (including, e.g., antibodies, antibody fragments, polypeptides, or Fc-containing molecules). Such an agent (which may also be referred to as a secondary molecule) may be, for example, a small molecule (e.g., not a protein or polypeptide), such as a small molecule drug, or may be a peptide, or may be a polypeptide, or may be an antibody or fragment thereof, or a plurality or combination of such molecules. Such an agent may be one that binds to the test molecule, or is suspected of binding to the test molecule; or regulates the binding of the test molecule to the receptor that mediates molecular transport, such as regulates the binding of the test molecule to FcRn; or is a ligand to a receptor of interest in the system being evaluated (e.g., a ligand to a receptor expressed by one or more cells of the cell layer); or is known or suspected of interacting with the test molecule, or a receptor expressed by the cell layer. The agent may be a therapeutic molecule, such as a prescription medication or an over the counter (OTC) medication; or a dietary supplement, a vitamin, a research reagent, an imaging reagent, or other agent of interest. Therapeutics may include those used to treat a disorder or condition of the immune system, cardiovascular system, endocrine system, gastrointestinal tract, renal system, respiratory system, or central nervous system, or to treat cancer or other malignancy (including chemotherapeutic and immunotherapy drugs), viral infection, bacterial infection, fungal infection, or a parasitic infection. Combinations of two or more agents of interest may also be evaluated. Thus, in some embodiments, the methods provided herein comprise evaluating the recycling, or recycling and transcytosis, of a test molecule in the presence of one or more agents. Said one or more agents may be included in the first chamber but not the second, or in the second chamber but not the first, or in both chambers, or is included in the first solution of the first chamber but then is not present when the solution is replaced, or combinations thereof. In some embodiments, the assay output is further compared to the recycling, or recycling and transcytosis, in the absence of the one or more agents. The ability to evaluate the effect of one or more agents on the recycling, or recycling and transcytosis, of a test molecule according to the methods and assays described herein may useful, for example, to determine how one or more drugs may affect one or more PK parameters of the test molecule, or may affect the tissue penetrance (e.g., brain penetrance) of the test molecule. The methods and assays described herein may, for example, be used to understand the impact of commonly co-administered drugs on the PK parameters and/or tissue penetrance of molecule of interest. The methods and assays described herein may, for example, be used to investigate the mechanisms of transcellular transport, PK parameters, and/or tissue penetrance.

The present disclosure also provides methods and assays for use in determining the effect of one or more changes in environmental conditions on the recycling, transcytosis, tissue penetrance, PK parameters, or other characteristic of a test molecule (including, e.g., antibodies, antibody fragments, polypeptides, or Fc-containing molecules). Such environmental conditions may include, for example, the presence, absence, or concentration of one or more cell medium components, or temperature, or pH, or oxidation, or combinations thereof. In some embodiments, the tissue penetrance is brain penetrance.

In certain embodiments, selection of the test molecule (including, e.g., antibodies, antibody fragments, polypeptides, or Fc-containing molecules) of the methods and assays of the present disclosure can be based on availability of the clinical-grade materials and the animal PK data and/or or animal tissue penetrance data from reliable sources such as, but not limited to, prescribing information, published reports based on population PK or tissue penetrance models or in-house clinical trials where lineal PK or tissue penetrance data were generated. In certain embodiments, the usage of clinical-grade materials can ensure that the quality of the test materials can be consistent with those used in clinical studies where the human PK or tissue penetrance data were generated.

In certain embodiments, methods and assays described in the present disclosure can be used as a tool for in vitro evaluation of potential liabilities in non-specific clearance of drug candidates (including, e.g., antibodies, antibody fragments, polypeptides, or Fc-containing molecules) to support lead selection and optimization, with the aim to rank order candidates and reduce the number of molecules tested in animal models, and/or reduce the number of animal models needed.

In certain embodiments, methods and assays described in the present disclosure can be used to support investigation of a molecule of interest (including, e.g., antibodies, antibody fragments, polypeptides, or Fc-containing molecules) exhibiting undesirable or atypical PK or tissue penetrance behavior, or development of novel drugs (including, e.g., antibodies, antibody fragments, polypeptides, or Fc-containing drugs) with improved recycling property pertinent to specialized applications such as crossing of blood-brain barrier for enhanced brain exposure or enhanced disposition in tumor microenvironment for improved tumor targeting. Such “improved recycling property” may include an increase of recycling compared to another molecule, which may in some embodiments increase serum half-life. In certain embodiments, methods and assays described in the present disclosure can be used to support investigation of a molecule of interest (including, e.g., antibodies, antibody fragments, polypeptides, or Fc-containing molecules) exhibiting undesirable or atypical PK behavior, or development of novel drugs with improved transcytosis property pertinent to specialized applications such as crossing of blood-brain barrier for enhanced brain exposure or enhanced disposition in tumor microenvironment for improved tumor targeting. Such “improved transcytosis property” may include an increase of transcytosis compared to another molecule. Still further, in certain embodiments the methods and assays described in the present disclosure can be used to support investigation of a molecule of interest (including, e.g., antibodies, antibody fragments, polypeptides, or Fc-containing molecules) exhibiting undesirable or atypical PK behavior, or development of novel drugs with improved transcytosis property pertinent to specialized applications such as crossing of blood-brain barrier for enhanced brain exposure or enhanced disposition in tumor microenvironment for improved tumor targeting. Such “improved transcytosis property” may include an increase of transcytosis compared to another molecule. Further contemplated is use of the assays described herein to support development of novel drugs (including, e.g., antibodies, antibody fragments, polypeptides, or Fc-containing molecules) with an improved transcytosis to recycling ratio, compared to another molecule. Such “improved transcytosis to recycling ratio” (T/R ratio) may include a higher ratio than another molecule, for example due to increased transcytosis, decreased recycling, or both. A higher T/R ratio may lead to better tissue penetrance, such as better brain penetrance, compared to another molecule. In some embodiments, the molecule of interest and/or drug is a mAb.

In certain embodiments, methods and assays described in the present disclosure can be used for the development of improved mechanism-based PK models to support design of optimal dose and dosing schemes in clinical studies. In certain embodiments, methods and assays described in the present disclosure can be used to demonstrate a correlation between an in vitro readout and in vivo PK data for test molecules(including, e.g., antibodies, antibody fragments, polypeptides, or Fc-containing molecules). Such PK models and/or data may involve in vivo clearance (CL), in vivo half-life (t½), area under the curve (AUC), bioavailability, volume of distribution (Vd), or maximum/minimum plasma concentrations (Cmax/Cmin).

In certain embodiments, the molecules of interest, test molecules and/or plurality of molecules can be antibodies. In certain embodiments, the antibodies can be monoclonal or polyclonal antibodies. In certain embodiments, the antibodies may have different structural composition (chimeric, humanized, and fully human). In certain embodiments, the antibodies may consist of varying heavy and light chain sequences (IgG1, IgG2, and IgG4 heavy chains, kappa and lambda light chains). In certain embodiments, the antibodies may recognize different type of targets (membrane bound and soluble). In certain embodiments, the antibodies may exert different therapeutic mechanism of action (agonistic, antagonistic, and cytotoxic). In certain embodiments, the antibodies may and are given to patients via different routes (e.g., intravenously, intramuscularly, or subcutaneously). In certain embodiments, the antibody is an anti-IgE antibody, an anti-VEGF antibody, an anti-integrin antibody (e.g., anti-α4β7 integrin antibody), anti-IL-6 antibody, anti-TNFa antibody, anti-BACE1 antibody, or anti-gD antibody, or a fragment thereof. In some embodiments, the antibody is omalizumab, bevacizumab, vedolizumab, tocilizumab, adalimumab, anti-BACE1 WT, anti-BACE1 variant YEY (mutations: M252Y; N286E; N434Y), anti-BACE1 variant YQAY (mutations: M252Y; T307Q; Q311A; N434Y), anti-BACE1 variant YPY (mutations: M252Y; V308P; N434Y), anti-BACE1 variant YY (mutations: M252Y; N434Y), anti-BACE1 variant YLY1 (Q6) (mutations: M252Y; M428L; N434Y; Y436I), or anti-BACE1 variant YIY (T3) (mutations: M252Y; Q3111; N434Y), or a fragment thereof. In some embodiments, two or more antibodies are evaluated.

In certain embodiments, the molecules of interest, test molecules, and/or plurality of molecules can carry engineered mutations that may alter their effector functions (such as but not limited to, atezolizumab, durvalumab), enable association of bi-specific half antibodies (such as, but not limited to, emicizumab), or stabilize IgG4 Fab arms (such as, but not limited to, nivolumab, pembrolizumab).

In certain embodiments, the molecules of interest, test molecules, and/or plurality of molecules can be antibodies exhibiting extreme FcRn binding affinities. In certain embodiments, the observed correlation between recycling readout and PK parameter in a mammal (such as a non-human primate, or a human) may apply to a broad range of antibodies carrying typical Fc regions. In certain embodiments, the test molecules may be antibodies that are engineered to have altered FcRn binding activity. In certain embodiments, the molecules of interest, test molecules and/or plurality of molecules may be albumin-containing molecules, or any molecules engineered to bind to FcRn via peptide tags or recombinant proteins. In certain embodiments, the molecules of interest, test molecules and/or plurality of molecules can be molecules that naturally bind to FcRn. In certain embodiments, the molecules of interest, test molecules and/or plurality of molecules can be molecules engineered to bind to FcRn.

In certain embodiments, the methods and assays described in the present disclosure may be used to indicate whether or not the expression of human FcRn is required for efficient recycling, and in some embodiments recycling and transcytosis, in a recycling or recycling and transcytosis assay.

In certain embodiments, the methods and assays of the present disclosure may be used to detect and/or indicate that interactions with FcRn contribute to the observed correlation between the recycling readout and PK parameter of the test molecules. In some embodiments, the in vivo clearance (CL), in vivo half-life (t½), area under the curve (AUC), bioavailability, volume of distribution (Vd), or maximum/minimum plasma concentrations (Cmax/Cmin).

In certain embodiments, the methods and assays of the present disclosure may be used for the binding analysis of the molecules of interest, test molecules and/or plurality of molecules to a receptor. In certain embodiments, the receptor can be the FcRn receptor. In certain embodiments, the test molecule can be a an antibody. In certain embodiments, the receptor can be the transferrin receptor. In certain embodiments, the receptor is a human, mouse, rat, or cynomolgus transferrin receptor. In certain embodiments, the receptor is a transferrin 1 or transferrin 2 receptor. In certain embodiments, the receptor is a transferrin receptor described in UNIPROT P02786 (TFR1_HUMAN), UNIPROT Q9UP52 (TFR2_HUMAN), UNIPROT A0A2K5X958 (A0A2K5X958_MACFA, cyno), UNIPROT Q62351 (TFR1_MOUSE), UNIPROT Q9JKX3 (TFR2_MOUSE), UNIPROT Q99376 (TFR1_RAT), or UNIPROT B2GUY2 (TFR2_RAT). In some embodiments, the receptor is described in UNIPROT P02786 (TFR1_HUMAN), or UNIPROT Q9UP52 (TFR2_HUMAN). In certain embodiments, the receptor can be the megalin receptor. In certain embodiments, the receptor is a megalin receptor described in UNIPROT P98164 (LRP2_HUMAN), UNIPROT A2ARV4 (LRP2_MOUSE), or UNIPROT P98158 (LRP2_RAT). In certain embodiments, the receptor can be the cubulin receptor. In some embodiments, the receptor is a cubulin receptor described in UNIPROT 060494 (CUBN_HUMAN), UNIPROT Q9JLB4 (CUBN_MOUSE), UNIPROT 070244 (CUBN_RAT), or UNIPROTA0A2K5USK6 (A0A2K5USK6_MACFA, cyno)

In certain embodiments, the FcRn receptor of the methods, assays, assay systems and kits of the present invention is a human FcRn receptor.

In certain embodiments, the heavy chain of the human FcRn receptor of the methods, assays, assay systems and kits of the present invention has the sequence:

(SEQ ID NO: 1)
AESHLSLLYHLTAVSSPAPGTPAFWVSGWLGPQQYLSYNSLRGEAEP
CGAWVWENQVSWYWEKETTDLRIKEKLFLEAFKALGGKGPYTLQGLL
GCELGPDNTSVPTAKFALNGEEFMNFDLKQGTWGGDWPEALAISQRW
QQQDKAANKELTFLLFSCPHRLREHLERGRGNLEWKEPPSMRLKARP
SSPGFSVLTCSAFSFYPPELQLRFLRNGLAAGTGQGDFGPNSDGSFH
ASSSLTVKSGDEHHYCCIVQHAGLAQPLRVELESPAKSSVLVVGIVI
GVLLLTAAAVGGALLWRRMRSGLPAPWISLRGDDTGVLLPTPGEAQD
ADLKDVNVIPATA.

In certain embodiments, the light chain of the human FcRn receptor of the methods, assays, assay systems and kits of the present invention has the sequence:

(SEQ ID NO: 2)
MSRSVALAVLALLSLSGLEAIQRTPKIQVYSRHPAENGKSNFLNCYV
SGFHPSDIEVDLLKNGERIEKVEHSDLSFSKDWSFYLLYYTEFTPTE
KDEYACRVNHVTLSQPKIVKWDRDM.

In certain embodiments, the FcRn receptor of the methods, assays, assay systems and kits of the present invention is a mouse FcRn receptor.

In certain embodiments, the heavy chain of the mouse FcRn receptor of the methods, assays, assay systems and kits of the present invention has the sequence:

(SEQ ID NO: 3)
SETRPPLMYHLTAVSNPSTGLPSFWATGWLGPQQYLTYNSLRQEADP
CGAWMWENQVSWYWEKETTDLKSKEQLFLEALKTLEKILNGTYTLQG
LLGCELASDNSSVPTAVFALNGEEFMKFNPRIGNWTGEWPETEIVAN
LWMKQPDAARKESEFLLNSCPERLLGHLERGRRNLEWKEPPSMRLKA
RPGNSGSSVLTCAAFSFYPPELKFRFLRNGLASGSGNCSTGPNGDGS
FHAWSLLEVKRGDEHHYQCQVEHEGLAQPLTVDLDSSARSSVPVVGI
VLGLLLVVVAIAGGVLLWGRMRSGLPAPWLSLSGDDSGDLLPGGNLP
PEAEPQGANAFPATS.

In certain embodiments, the light chain of the mouse FcRn receptor of the

methods, assays, assay systems and kits of the
present invention has the sequence:
(SEQ ID NO: 4)
MARSVTLVFLVLVSLTGLYAIQKTPQIQVYSRHPPENGKPNILNCYV
TQFHPPHIEIQMLKNGKKIPKVEMSDMSFSKDWSFYILAHTEFTPTE
TDTYACRVKHDSMAEPKTVYWDRDM.

In certain embodiments, the FcRn receptor of the methods, assays, assay systems and kits of the present invention is a rat FcRn receptor.

In certain embodiments, the heavy chain of the rat FcRn receptor of the methods, assays, assay systems and kits of the present invention has the sequence:

(SEQ ID NO: 5)
AEPRLPLMYHLAAVSDLSTGLPSFWATGWLGAQQYLTYNNLRQEADP
CGAWIWENQVSWYWEKETTDLKSKEQLFLEAIRTLENQINGTFTLQG
LLGCELAPDNSSLPTAVFALNGEEFMRFNPRTGNWSGEWPETDIVGN
LWMKQPEAARKESEFLLTSCPERLLGHLERGRQNLEWKEPPSMRLKA
RPGNSGSSVLTCAAFSFYPPELKFRFLRNGLASGSGNCSTGPNGDGS
FHAWSLLEVKRGDEHHYQCQVEHEGLAQPLTVDLDSPARSSVPVVGI
ILGLLLVVVAIAGGVLLWNRMRSGLPAPWLSLSGDDSGDLLPGGNLP
PEAEPQGVNAFPATS.

In certain embodiments, the light chain of the rat FcRn receptor of the methods, assays, assay systems and kits of the present invention has the sequence:

(SEQ ID NO: 6)
MARSVTVIFLVLVSLAVVLAIQKTPQIQVYSRHPPENGKPNFLNCYV
SQFHPPQIEIELLKNGKKIPNIEMSDLSFSKDWSFYILAHTEFTPTE
TDVYACRVKHVILKEPKTVTWDRDM.

In certain embodiments, the FcRn receptor of the methods, assays, assay systems and kits of the present invention is a cynomologus monkey (cyno) FcRn receptor.

In certain embodiments, the heavy chain of the cyno FcRn receptor of the methods, assays, assay systems and kits of the present invention has the sequence:

(SEQ ID NO: 7)
AESHLSLLYHLTAVSSPAPGTPAFWVSGWLGPQQYLSYDSLRGQAEP
CGAWVWENQVSWYWEKETTDLRIKEKLFLEAFKALGGKGPYTLQGLL
GCELSPDNTSVPTAKFALNGEEFMNFDLKQGTWGGDWPEALAISQRW
QQQDKAANKELTFLLFSCPHRLREHLERGRGNLEWKEPPSMRLKARP
GNPGFSVLTCSAFSFYPPELQLRFLRNGMAAGTGQGDFGPNSDGSFH
ASSSLTVKSGDEHHYCCIVQHAGLAQPLRVELETPAKSSVLVVGIVI
GVLLLTAAAVGGALLWRRMRSGLPAPWISLRGDDTGSLLPTPGEAQD
ADSKDINVIPATA.

In certain embodiments, the light chain of the cyno FcRn receptor of the methods, assays, assay systems and kits of the present invention has the sequence:

(SEQ ID NO: 8)
MSPSVALAVLALLSPSGLEAIQRTPKIQVYSRHPPENGKPNFLNCYV
SGFHPSDIEVDLLKNGEKMGKVEHSDLSFSKDWSFYLLYYTEFTPNE
KDEYACRVNHVTLSGPRTVKWDRDM.

In certain embodiments, methods, assays, assay systems and kits of the present invention can be used to evaluate the contribution of multiple factors, such as but not limited to, internalization, dynamics of sorting and intracellular trafficking, and exocytosis to the overall efficiency of the recovery process of the test molecule (including, e.g., antibodies, antibody fragments, polypeptides, or Fc-containing molecules). In certain embodiments, the methods and assays of the present disclosure can be used to identify and/or detect multiple parameters involved in the FcRn-mediated salvage pathway for determination or prediction of PK of the test molecule (including, e.g., antibodies, antibody fragments, polypeptides, or Fc-containing molecules).

In certain embodiments, the methods, assays, assay systems and kits of the present disclosure can be used to detect correlation of in vitro recycling and in vivo clearance of a test molecule (including, e.g., antibodies, antibody fragments, polypeptides, or Fc-containing molecules). In certain embodiments, the assays of the present disclosure can be used to detect correlation of in vitro recycling and in vivo clearance of a molecule as compared to BV ELISA or Fv charge/pI.

In certain embodiments, the methods, assays, assay systems and kits of the present disclosure can be used to evaluate and/or investigate the role of glycosylation on the distribution and catabolism of a test molecule (including, e.g., antibodies, antibody fragments, polypeptides, or Fc-containing molecules) in vivo. In certain embodiments, the molecule(s) can be glycoform variants.

In certain embodiments, the methods, assays, assay systems and kits of the present disclosure can be used to evaluate the role of the addition of sialic acid to the molecule(s) on intracellular trafficking parameters. In certain embodiments, they can be used for the analysis of the involvement of additional mechanisms in clearance of high-mannose and highly sialylated glycoform variants of the molecule(s) in vivo.

In certain embodiments, the methods, assays, assay systems and kits of the present disclosure can be used as a tool for the study of the FcRn-mediated transcytosis and recycling as an elimination mechanism of a test molecule (including, e.g., antibodies, antibody fragments, polypeptides, or Fc-containing molecules) pertaining to their PK.

In certain embodiments, the methods, assays, assay systems and kits of the present disclosure can be used as a tool for the study and the elucidation of the molecular mechanism governing distribution of FcRn-complexed IgGs.

In certain embodiments, the methods, assays, assay systems and kits of the present disclosure can to provide an output that reflects the target-independent, non-specific clearance mechanism of a test molecule (including, e.g., antibodies, antibody fragments, polypeptides, or Fc-containing molecules) with typical FcRn binding affinity in humans.

In certain embodiments, the methods, assays, assay systems and kits of the present disclosure can be used for the analysis of a diverse group of Fc-containing molecules and respond to factors known to impact PK.

In certain embodiments, the methods, assays, assay systems and kits of the present embodiment can be used to dissect factors involved in recycling assays, and recycling and transcytosis assays, in order to understand how these factors correlate with in vivo clearance and to define the quantitative nature of this correlation.

In certain embodiments, the methods, assays, assay systems and kits of the present embodiment can be used to dissect factors involved in recycling assays, and recycling and transcytosis assays, in order to understand how these factors correlate with tissue penetrance and to define the quantitative nature of this correlation. In some embodiments, the tissue penetrance is brain penetrance.

In certain embodiments, the methods, assays, assay systems and kits of the present disclosure can be used for the investigation of the distribution of pinocytosed or internalized test molecule (including, e.g., antibodies, antibody fragments, polypeptides, or Fc-containing molecules) among recycling, transcytosis, and degradation pathways in various cell type/tissue compartments, as well as the molecular mechanisms governing such distributions in cells involved in the clearance of IgG.

In certain embodiments, the present disclosure is directed to and recycling assay systems. In certain embodiments, the present disclosure is directed to and recycling and transcytosis assay systems. For example, but not by way of limitation, such systems can comprise: a first chamber and a second chamber, wherein each of the first and second chambers comprise aqueous solution at physiological pH; a cell layer separating the first and second chambers such that the cell layer can mediate the recycling of a molecule introduced into the first chamber into the cell layer and back to the first chamber; and a detector for detecting the presence of a molecule in the first chamber. In certain embodiments, the assay systems disclosed herein employ a cell layer of eukaryotic cells, such as mammalian cells, for example rodent, canine, non-human primate, or human cells. In some embodiments, the assay systems disclosed herein employ a cell layer of kidney cells. In some embodiments, the assays systems disclosed herein employ a cell layer of MDCK cells. In some embodiments, the cell layer is a monocellular layer. In certain embodiments, the assay systems disclosed herein employ cells comprising at least one heterologous gene. In certain embodiments, the assay systems disclosed herein employ cells comprising at least one heterologous gene selected from the group consisting of a FCGRT gene and a B2M gene. In certain embodiments, the assay systems disclosed herein employ cells that express a heterologous cell surface protein. In certain embodiments, the assay systems disclosed herein employ cells expressing a heterologous cell surface protein where the heterologous cell surface protein comprises a neonatal Fc receptor (FcRn). In certain embodiments, the assay systems disclosed herein can measure the number of the recycled molecules via use of an enzyme-linked immunosorbent assay (ELISA), liquid-scintillation counting (LSC), quantitative PCR, a fluorescence reader system, automated immunoassay platforms (e.g. Quanterix, Gyrolab, and Singulex), fluorescence imaging, or mass spectrometry. In certain embodiments, the assay systems disclosed herein can measure the number of the recycled molecules using a method with an LLOQ of at least 200 pg/mL, or at least 100 pg/mL, or at least 50 pg/mL, such as an ELISA assay using a high-affinity capture reagent (e.g., a biotinylated mouse anti-human IgG-Fc, such as biotin-R10Z). In certain embodiments, in a recycling and transcytosis assay system of the present disclosure, the cell layer can mediate the transcytosis of a molecule from the first chamber to the second chamber. In some embodiments, the assay system further comprises a detector for detecting the presence of a molecule in the second chamber. Such a detector may the same type and/or have the same LLOQ as a detector for detecting the presence of a molecule in the first chamber, or it may be of another type and/or difference sensitivity, for example a less sensitive type (e.g., have a LLOQ in the nanomolar concentration range).

### 4. Cells

Suitable cells to be used in the methods, assays, assay systems or kits of the present disclosure, include prokaryotic or eukaryotic cells as described herein.

In certain embodiments, vertebrate cells can also be used. Non-limiting examples of useful mammalian cell lines are monkey kidney CV1 line transformed by SV40 (COS-7); primary human endothelial cells; human umbilical vein endothelial cells (HUVECs); human brain microvascular endothelial cells (HBMEC); vascular endothelial cells (EA.hy926); human dermal microvascular endothelial cells (HMEC); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Additional non-limiting examples of useful mammalian cell lines include Chinese hamster ovary (CHO) cells, including DHFR-CHO cells (Urlaub et al., Proc. Natl. Acad. Sci. USA 77:4216 (1980)) In certain embodiments, the methods of the present disclosure can use hybridoma cells. For example, but not by way of limitation, the hybrid cell line can be of any species, including human and mouse. In certain embodiments, the methods of the present disclosure can use primary and established endothelial and/or epithelial cells. For example, but not by way of limitation, the endothelial and/or epithelial cells can be caco-2, T-84, HMEC-1, MHEC 2.6, HUVEC, and induced pluripotent stem cell (iPSC)-derived cells (see, e.g., Lidington, E. A., D. L. Moyes, et al. (1999), “A comparison of primary endothelial cells and endothelial cell lines for studies of immune interactions” Tramp' Immunol 7(4): 239-246.; or Yamaura, Y., B. D. Chapron, et al. (2016), “Functional Comparison of Human Colonic Carcinoma Cell Lines and Primary Small Intestinal Epithelial Cells for Investigations of Intestinal Drug Permeability and First-Pass Metabolism,” Drug Metab Dispos 44(3): 329-335).

In certain embodiments, a cell for use in the disclosed methods, assays, assay systems or kits can comprise a nucleic acid that encodes a molecule, e.g., a receptor. In certain embodiments, the nucleic acid can be present in one or more vectors, e.g., expression vectors. One type of vector is a “plasmid,” which refers to a circular double stranded DNA loop into which additional DNA segments can be ligated. Another type of vector is a viral vector, where additional DNA segments can be ligated into the viral genome. Certain vectors are capable of autonomous replication in a cell into which they are introduced (e.g., bacterial vectors having a bacterial origin of replication and episomal mammalian vectors). Other vectors (e.g., non-episomal mammalian vectors) are integrated into the genome of a cell upon introduction into the cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). Additional non-limiting examples of expression vectors for use in the present disclosure include viral vectors (e.g., replication defective retroviruses, adenoviruses and adeno-associated viruses) that serve equivalent functions.

In certain embodiments, the nucleic acid encoding a molecule, e.g., a receptor, can be introduced into a cell. In certain embodiments, the introduction of a nucleic acid into a cell can be carried out by any method known in the art including, but not limited to, transfection, electroporation, microinjection, infection with a viral or bacteriophage vector containing the nucleic acid sequences, cell fusion, chromosome-mediated gene transfer, microcell-mediated gene transfer, spheroplast fusion, etc. In certain embodiments, the cell is eukaryotic, e.g., a MDCK cell.

### 5. Chambers

The methods, assays, assay systems, or kits of the present disclosure require a first chamber and a second chamber, wherein the first and second chambers are separated by a cell layer. Any suitable configuration of chambers that are capable of retaining aqueous solutions in two separate compartments that intersect along a cell layer may be used. Materials for said chambers may include, for example, plastic (e.g., polycarbonate), glass, ceramic, coated materials (e.g., plasticized metal), or mixtures thereof. Said chambers may be fully enclosed, or may have one or more sides exposed, as long as they can retain sufficient aqueous solution in both chambers and maintain a cell layer separating said chambers. Said chambers may further include configurations wherein one chamber is of smaller volume than the other chamber, and for example fits within the three dimensional space of the other chamber (e.g., is an insert), separated by the cell layer. In some embodiments, the smaller chamber (which may also be described as the “inner chamber”) is the first chamber, the larger chamber (which may also be described as the “outer chamber”) is the second chamber. However, such terminology is not meant to limit the use of the presently described assays and systems to alternative chamber combinations (e.g., the outer chamber may be used as a first chamber, and inner chamber as a second chamber). The test molecule is introduced into the first chamber, in whichever configuration of chambers is used. In some embodiments, the chambers are trans-well systems. Suitable trans-well systems that can be employed in the methods, assays, assay systems or kits of the present disclosure include but are not limited to, Boyden chambers, cell migration assays, cell invasion assays, microfluidic migration devices, in vitro scratch assays, extracellular matrix (ECM) proteins assays. The methods of the present disclosure can be conducted utilizing a broad variety of assay platforms (e.g., 12-well, 24-well or 96-well multi-well arrays), including “generic” trans-well platforms. Non-limiting examples of assay platforms include MILLICELL® cell culture inserts and insert plates, and CORNING® TRANSWELL® polycarbonate membrane cell culture inserts.

### EXEMPLARY EMBODIMENTS

E1. A method, comprising determining the recycling of a plurality of molecules, wherein the determining comprises:
   introducing the plurality of molecules into a first chamber, wherein:
      the first chamber is separated from a second chamber by a cell layer, and
      the first and second chambers comprise an aqueous solution;
   incubating the plurality of molecules in the first chamber;
   replacing the aqueous solution in both the first and second chambers; and
   measuring the amount of the plurality of molecules that is released from the cell layer into the first chamber;
   wherein the aqueous solution is at physiological pH, and the cell layer comprises cells that express a receptor that mediates molecular transport.
E2. The method of E1, wherein the plurality of molecules is a plurality of a single molecule.
E3. The method of E1, wherein the plurality of molecules is a plurality of distinct molecules.
E4. The method of any one of E1 to E3, wherein the cell layer comprises a cell monolayer.

E5. The method of any one of E1 to E4, wherein the receptor that mediates molecular transport is a receptor that mediates intracellular transport of molecules.
E6. The method of any one of E1 to E4, wherein the receptor that mediates molecular transport is a transferrin receptor, an Fc receptor, megalin, or cubulin.
E7. The method of any one of E1 to E6, wherein the receptor that mediates molecular transport is a neonatal Fc receptor (FcRn).
E8. The method of any one of E1 to E7, wherein the cells are eukaryotic cells or mammalian cells.
E9. The method of any one of E1 to E7, wherein the cells are Madin-Darby Canine Kidney (MDCK) cells.
E10. The method of any one of E1 to E9, wherein measuring the amount of the plurality of molecules that is released from the cell layer comprises the use of an enzyme-linked immunosorbent assay (ELISA), liquid-scintillation counting (LSC), quantitative PCR, a fluorescence reader system, or mass spectrometry.
E11. The method of any one of E1 to E1 0, wherein the plurality of molecules is incubated in the first chamber from about 1 hour to about 48 hours.
E12. The method of any one of E1 to E11, wherein the plurality of molecules is incubated in the first chamber from about 1 hour to about 30 hours.
E13. The method of any one of E1 to E12, wherein replacing the aqueous solution comprises washing the first chamber.
E14.The method of any one of E1 to E13, further comprising incubating the first and second chambers with the replacement aqueous solution prior to measuring.
E15. The method of E14, wherein the first and second chambers are incubated with the replacement aqueous solution for between 1 hour and 6 hours prior to measuring.
E16. The method of any one of E1 to E15, wherein the incubation is at a temperature of between about 35° C. to about 39° C.
E17. The method of any one of E1 to E15, wherein the plurality of molecules are Fc-containing molecules.
E18. The method of E17, wherein the Fc-containing molecules are receptor Fc fusion molecules.
E19. The method of any one of E1 to E15, wherein the plurality of molecules are antibodies.
E20. The method of E1 9, wherein the antibodies are monoclonal antibodies.
E21. The method of E20, wherein the antibody is an anti-IgE antibody, an anti-VEGF antibody, an anti-integrin antibody, an anti-IL-6 antibody, an anti-TNFa antibody, an anti-BACE1 antibody, or an anti-gD antibody.
E22. The method of E20, wherein the antibody is omalizumab, bevacizumab, tocilizumab, anti-BACE1 WT, anti-BACE1 variant YEY, anti-BACE1 variant YQAY, anti-BACE1 variant YPY, anti-BACE1 variant YY, anti-BACE1 variant YLY1 (Q6), or anti-BACE1 variant YIY (T3).
E23. The method of E7, wherein the FcRn is selected from the group consisting of human RcRn, mouse FcRn, rat FcRn, and cynomolgus FcRn.
E24. The method of any one of E1 to E23, wherein the physiological pH is about 7.4.
E25. The method of any one of E1 to E24, further comprising measuring the transcytosis of the plurality of molecules across the cell layer.
E26. The method of E25, wherein measuring the transcytosis comprises:
   after the aqueous solution has been replaced in both the first and second chambers, measuring the amount of the plurality of molecules in the second chamber.
E27. The method of any one of E1 to E26, comprising incubating the plurality of molecules in the first chamber in the presence of an agent, and determining whether the agent affects the recycling of the plurality of molecules.
E28. The method of E27, wherein the agent is a small molecule or polypeptide.
E29. A method of determining the tissue penetrance of a plurality of molecules, comprising:
   a) introducing the plurality of molecules into a first chamber, wherein the first chamber is separated from a second chamber by a cell layer, and the first and second chambers comprise an aqueous solution;
      wherein the aqueous solution is at physiological pH, and the cell layer comprises cells that express a receptor that mediates molecular transport;
   b) measuring the amount of the plurality of molecules that is recycled from the first chamber into the cell layer and back to the first chamber;
   c) measuring the amount of the plurality of molecules that is transcytosed from the first chamber to the second chamber; and
   d) determining the tissue penetrance of the plurality of molecules based on the ratio of transcytosed to recycled molecules.
E30. The method of E29, wherein measuring the plurality of molecules that is recycled comprises:
   after introducing the plurality of molecules into the first chamber, incubating the plurality of molecules in the first chamber;
   replacing the aqueous solution in both the first and second chambers; and
   measuring the amount of the plurality of molecules that is released from the cell layer into the first chamber.
E31. The method of E29 or E30, wherein measuring the plurality of molecules that is transcytosed comprises:
   after introducing the plurality of molecules into the first chamber, incubating the plurality of molecules in the first chamber;
   replacing the aqueous solution in both the first and second chambers; and
   measuring the amount of the plurality of molecules that is released from the cell layer into the second chamber.
E32. The method of E30 or E31, wherein the plurality of molecules is incubated in the first chamber from at least about 1 hour to about 48 hours.
E33. The method of E30 or E31, wherein the plurality of molecules is incubated in the first chamber from about 1 hour to about 30 hours.
E34. The method of any one of E30 to E33, wherein replacing the aqueous solution comprises washing the first chamber.
E35. The method of any one of E29 to E34, further comprising incubating the first and second chambers with the replacement aqueous solution prior to measuring.
E36. The method of E35, wherein the first and second chambers are incubated with the replacement aqueous solution between 1 hour and 6 hours prior to measuring.
E37. The method of any one of E29 to E36, wherein the incubation is at a temperature of between about 35° C. to about 39° C.
E38. The method of any one of E29 to E36, wherein the plurality of molecules is a plurality of a single molecule.
E39. The method of any one of E29 to E36, wherein the plurality of molecules is a plurality of distinct molecules.
E40. The method of any one of E29 to E39, wherein the cell layer comprises a cell monolayer.
E41. The method of any one of E29 to E40, wherein the receptor that mediates molecular transport is a receptor that mediates intracellular transport of molecules.
E42. The method of any one of E29 to E41, wherein the receptor that mediates molecular transport is a transferrin receptor, an Fc receptor, megalin, or cubulin.
E43. The method of any one of E29 to E42, wherein the receptor that mediates molecular transport is a neonatal Fc receptor (FcRn).
E44. The method of any one of E29 to E43, wherein the cells are eukaryotic cells or mammalian cells.
E45. The method of any one of E29 to E44, wherein the cells are Madin-Darby Canine Kidney (MDCK) cells.
E46. The method of any one of E29 to E45, wherein measuring the amount of the plurality of molecules that is recycled and measuring the amount of the plurality of molecules that is transcytosed independently comprise the use of an enzyme-linked immunosorbent assay (ELISA), liquid-scintillation counting (LSC), quantitative PCR, a fluorescence reader system, mass spectrometry, or any combinations thereof.
E47. The method of any one of E29 to E46, wherein the plurality of molecules are Fc-containing molecules.
E48. The method of E47, wherein the Fc-containing molecules are receptor Fc fusion molecules.
E49. The method of any one of E29 to E48, wherein the plurality of molecules are antibodies.
E50. The method of E47, wherein the antibodies are monoclonal antibodies.
E51. The method of E50, wherein the antibody is an anti-IgE antibody, an anti-VEGF antibody, an anti-integrin antibody, an anti-IL-6 antibody, an anti-TNFa antibody, an anti-BACE1 antibody, or an anti-gD antibody.
E52. The method of E50, wherein the antibody is omalizumab, bevacizumab, tocilizumab, anti-BACE1 WT, anti-BACE1 variant YEY, anti-BACE1 variant YQAY, anti-BACE1 variant YPY, anti-BACE1 variant YY, anti-BACE1 variant YLY1 (Q6), or anti-BACE1 variant YIY (T3).
E53. The method of any one of E29 to E52, wherein the physiological pH is about 7.4.
E54. The method of any one of E29 to E53, wherein the tissue penetrance is brain penetrance.
E55. The method of any one of E29 to E54, comprising incubating the plurality of molecules in the first chamber in the presence of an agent, and determining whether the agent affects the tissue penetrance of the plurality of molecules.
E56. The method of E55, wherein the agent is a small molecule.
E57. A method of determining a pharmacokinetic (PK) parameter of a plurality of molecules, comprising:
   a) introducing the plurality of molecules into a first chamber, wherein the first chamber is separated from a second chamber by a cell layer, and the first and second chambers comprise an aqueous solution;
      wherein the aqueous solution is at physiological pH, and the cell layer comprises cells that express a receptor that mediates molecular transport;
   b) measuring the amount of the plurality of molecules that is recycled from the first chamber into the cell layer and back to the first chamber; and
   c) determining the PK parameter based on the amount of the plurality of molecules that is recycled.
E58. The method of E57, wherein measuring the plurality of molecules that is recycled comprises:
   after introducing the plurality of molecules into the first chamber, incubating the plurality of molecules in the first chamber;
   replacing the aqueous solution in both the first and second chambers; and
   measuring the amount of the plurality of molecules that is released from the cell layer into the first chamber.
E59. The method of E58, wherein the plurality of molecules is incubated in the first chamber from at least about 1 hour to about 48 hours.
E60. The method of E58, wherein the plurality of molecules is incubated in the first chamber from about 1 hour to about 30 hours.
E61. The method of any one of E58 to E60, wherein replacing the aqueous solution comprises washing the first chamber.
E62. The method of any one of E54 to E57, further comprising incubating the first and second chambers with the replacement aqueous solution prior to measuring.
E63. The method of E62, wherein the first and second chambers are incubated with the replacement aqueous solution for at least about 1 hour to about 6 hours prior to measuring.
E64. The method of any one of E58 to E63, wherein the incubation is at a temperature of between about 35° C. to about 39° C.
E65. The method of any one of E57 to E63, wherein the plurality of molecules is a plurality of a single molecule.
E66. The method of any one of E57 to E64, wherein the plurality of molecules is a plurality of distinct molecules.
E67. The method of any one of E57 to E66, wherein the cell layer comprises a cell monolayer.
E68. The method of any one of E57 to E67, wherein the cells express a heterologous FcRn.
E69. The method of any one of E57 to E68, wherein the cells are Madin-Darby Canine Kidney (MDCK) cells.
E70. The method of any one of E57 to E68, wherein measuring the amount of the plurality of molecules that is recycled comprises the use of an enzyme-linked immunosorbent assay (ELISA), liquid-scintillation counting (LSC), quantitative PCR, a fluorescence reader system, or mass spectrometry.
E71. The method of any one of claims 57 to 70, wherein the plurality of molecules are Fc-containing molecules.
E72. The method of E71, wherein the Fc-containing molecules are receptor Fc fusion molecules.
E73. The method of any one of E57 to E72, wherein the plurality of molecules are antibodies.
E74. The method of E73, wherein the antibodies are monoclonal antibodies.
E75. The method of E74, wherein the antibody is an anti-IgE antibody, an anti-VEGF antibody, an anti-integrin antibody, an anti-IL-6 antibody, an anti-TNFa antibody, an anti-BACE1 antibody, or an anti-gD antibody.
E76. The method of E74, wherein the antibody is omalizumab, bevacizumab, tocilizumab, anti-BACE1 WT, anti-BACE1 variant YEY, anti-BACE1 variant YQAY, anti-BACE1 variant YPY, anti-BACE1 variant YY, anti-BACE1 variant YLY1 (Q6), or anti-BACE1 variant YIY (T3).
E77. The method of any one of E57 to E76, wherein the physiological pH is about 7.4.
E78. The method of any one of E57 to E77, wherein the PK parameter is a measure of in vivo clearance, volume of distribution, area under the curve (AUC), or in vivo half-life of the plurality of molecules.
E79. The method of any one of E57 to E78, comprising incubating the plurality of molecules in the first chamber in the presence of an agent, and determining whether the agent affects the PK parameter of the plurality of molecules.
E80. The method of E79, wherein the agent is a small molecule.
E81. An assay system, comprising:
   a) a first chamber and a second chamber, wherein each chamber comprises aqueous solution at physiological pH;
   b) a cell layer separating the first and second chamber, wherein the cell layer can mediate recycling of a molecule from the first chamber, into the cell layer, and back into the first chamber;
   c) a detector for detecting the presence of a molecule in the first chamber;
wherein the assay system is configured to determine the recycling of a plurality of molecules, wherein the determining comprises:
   introducing the plurality of molecules into the first chamber;
   incubating the plurality of molecules in the first chamber;
   replacing the aqueous solution in both the first and second chambers; and
      measuring the amount of the plurality of molecules that is released from the cell layer into the first chamber.
E82. The assay system of E81, wherein:
   the assay system further comprises a detector for detecting the presence of a molecule in the second chamber;
   the cell layer can mediate the transcytosis of a molecule from the first chamber to the second chamber; and
   wherein the assay system is configured to determine the transcytosis of a plurality of molecules across the cell layer, wherein determining transcytosis comprises, after the aqueous solution has been replaced, measuring the amount of the plurality of molecules in the second chamber.
E83. The assay system of E81 or E82, wherein the first and second chambers are components of a 96-well trans-well plate.
E84. A kit, comprising the assay system of any one of E81 to E83, and instructions for use.

### EXAMPLES

The following examples are merely illustrative of the presently disclosed subject matter and should not be considered as limitations in any way.

### Materials and Methods

### MDCK Cell Line Expressing Human FcRn

A clonal cell line stably expressing both human FcRn heavy chain (FCGRT) and light chain (B2M) was generated as published previously (Chung, S. et al., 2019, MAbs, 11(5), 942-955). Briefly, the cDNA of the two genes FCGRT (UniProtKB-P55899, FCGRTN_HUMAN) and B2M (UniProtKB-P61769, B2MG_HUMAN) were introduced to the MDCK cells with a modified pPRK plasmid, then selected with puromycin and sorted by fluorescence-activated cell sorting (FACS). The final clone used for the assay was chosen based on significant expression level of both FCGRT and B2M proteins. MDCK-hFcRn cells were maintained in Dulbecco's modified minimal essential media (DMEM) with GlutaMAX (Thermo Fisher Scientific, Waltham, MA) supplemented with 10% FBS (HyClone, Logan, UT), 5 μg/mL puromycin and 1% Penicillin-Streptomycin (Thermo Fisher Scientific). The cells were cultured at 37° C. in 5% CO₂ and were passaged every two to three days.

### Test Molecules

The test molecules included a set of humanized IgG1 antibodies—a panel of seven therapeutic antibodies with documented or published clearance values in human studies, and anti-herpes simplex virus glycoprotein D wildtype (anti-gD WT) with two of its variants with engineered mutations that alters Fc-FcRn binding affinity (anti-gD HAHQ and anti-gD YTE). Among the 7 therapeutic antibodies, 5 of them are marketed drug products, of which 2 (vedolizumab and adalimumab) were purchased from the manufacturers. The remaining therapeutic antibodies (bevacizumab, omalizumab, tocilizumab, mAbX, and mAbY), along with the anti-gD variants were produced in engineered Chines Hamster Ovary (CHO) cells at Genentech (South San Francisco, CA).

### Recycling Assay

MDCK-hFcRn cells were seeded at a density of 6×10⁴ cells/well to the inner chamber of 96-well transwell plates (MilliporeSigma, Burlington, MA) with 100 μL growth medium in the inner chamber and 200 μL growth medium in the outer chamber, and were cultured three days to reach confluence on the membrane filter. To measure recycling, the medium in the inner chamber was removed and 50 μL test antibodies were loaded to each inner chamber at a concentration of 100 μg/mL (0.67 μM) followed by 2 hours incubation at 37° C. in 5% CO₂. The transwells were washed with growth medium to remove residual antibodies in both chambers, leaving 100 μL/well fresh cell culture medium in the inner chamber and 200 μL/well medium in the outer chamber. The cells were incubated for additional 4 hours at 37° C. in 5% CO₂, and the recycling samples were taken from inner chambers for the following quantification with a human IgG specific ELISA assay. In the last step of the experiment, the integrity of the cell monolayer was assessed with Lucifer Yellow (Sigma Aldrich, St. Louis, MO) by adding 20 mM Lucifer Yellow into the inner chamber and incubating for one hour. Samples taken from the outer chambers were then measured for their levels of Lucifer Yellow, and the results from wells with greater than 0.1% passive passage (measured by dividing Lucifer Yellow signal from outer chamber to that of the corresponding inner chamber) were considered invalid and discarded due to the potential leak of the cell monolayers. Each recycling assay measurement was done in 6-8 replicates and the reported value is the average concentration, normalized by the recycling output of bevacizumab tested in parallel in the same transwell plate.

Samples taken from the inner chambers represent the recycling component, and samples from the outer chambers represent the transcytosis component. The samples were frozen in −70° C. for quantification using ELISA.

Variations of this procedure may be used. For example, optionally, for the washing procedure for recycling component (inner chamber) the lowest speed of an electronic pipette can be used to minimize disturbance/damage to the cell layer cultured on the filter. Optionally, the double-digit lower limit of quantification of the immunoassay can be used to ensure the sensitivity is stringent enough to detect low concentration of antibodies. Optionally, the cells can be cultured under conditions to achieve a viability of over 95%. Optionally, cell culture medium may be adjusted depending on the type of cells being used, and/or the test molecule being evaluated. Optionally, large transwell plates may be used (e.g., 12-well or 24-well). Additional variations may be made.

### ELISA Method

A semi-homogenous bridging enzyme-linked immunosorbent assay (ELISA) using a biotinylated mouse anti-human IgG-Fc as the capture molecule (biotin-R10Z, prepared by Genentech), horseradish peroxidase (HRP)-conjugated goat anti-human Fab Jackson ImmunoResearch Laboratories, West Grove, PA) as the detection molecule, and 3,3′,5,5′-tetramethylbenzidine (TMB) solution (Kirkegaard & Perry Laboratories, Gaithersburg, MD) for color development, was used to evaluate samples. Specifically, standards, controls, and samples were diluted in the cell culture medium to reach their final concentrations, and other reagents were prepared in assay diluent (PBS/0.5% BSA/0.05% polysorbate 20/0.05% ProClin 300, pH 7.4±0.1). Each therapeutic molecule standard was serially diluted from 4 ng/mL to 31.25 pg/mL (in-well concentration). For the assay, a 60 μL/well of diluted standards, controls, or samples were incubated overnight at ambient temperature with 60 μL/well of 1 pg/mL of biotin-R10Z in a sealed 96-well polypropylene plate with gentle agitation. After overnight incubation, 100 μL/well of the mixture was added to the streptavidin-coated and pre-blocked plate (Roche Diagnostics Corporation, Indianapolis, IN). The plate was sealed and incubated for 1 hour at ambient temperature with agitation. The plate was washed 4 times with 400 μL/well per cycle of wash buffer (PBS/0.05% polysorbate 20, pH 7.34). Next, 100 μL/well of 0.1 μL/well HRP-conjugated goat anti-human was added and the plate sealed and incubated for 1 hour at ambient temperature. After washing the plate 4 times with 300 4/well per cycle wash buffer, 100 μL/well of peroxidase substrate solution B and TMB peroxidase substrate mixture (Kirkegaard & Perry Laboratories, Gaithersburg, MD) was added and the plate incubated for 15 to 20 minutes for color development. To stop color development, 100 μL of 1 M H₃PO₄ was added to each well. The plate was read on a SpectraMax 340 plate reader (Molecular Devices, San Jose, CA) at 450 nm (detection wavelength) with a 630 nm reference filter. Data were analyzed using 4-parameter fit and weighted with 1/y2 in SoftMax Pro 6.5.1 software program.

### Example 1: Development of the Recycling Assay

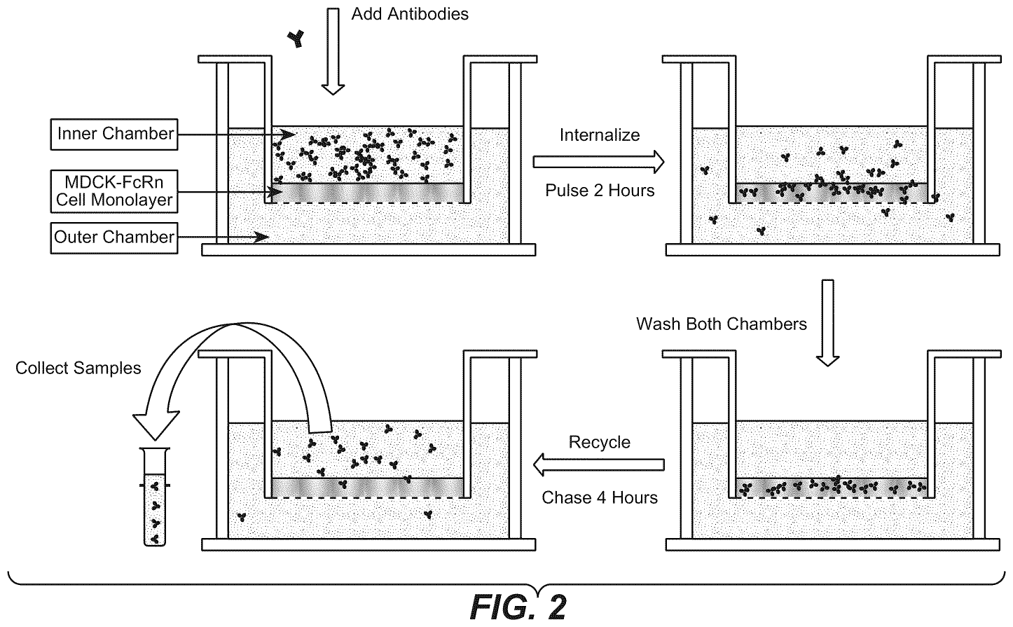

A clonal MDCK cell line (MDCK-hFcRn) engineered to express both human FcRn heavy chain (FCGRT) and light chain (B2M) was used to develop a FcRn-dependent recycling assay. Expression levels, subcellular colocalization and functional readout were demonstrated previously, suggesting functional human FcRn in the MDCK cell line (Chung, S. et al., 2019, MAbs, 11(5), 942-955). The recycling assay was designed in a pulse-chase experimental procedure (FIG. 2), and in the transwell culturing system which allows more complete apico-basolateral polarization of the cells. MDCK-hFcRn cells were seeded into 96-well trans-well plates and cultured for three days until confluence. For the pulse step, antibodies were added to the inner chamber and incubated for two hours to provide time for the antibodies to be internalized by the cells and to distribute and reach homeostatis among the intracellular pathways (recycling, transcytosis and degradation). After washing both the inner and outer chambers to remove the existing or transcytosed antibodies, fresh medium was added to both chambers and the cells were incubated for 4 hours. During this chase step, three possible outcomes were possible: the antibodies inside of the cells will either be recycled back into the inner chamber, transcytosed into the outer chamber, or degraded intracellularly. The antibodies that appear in the inner chamber represent the recycled molecules. Thus, the inner chamber solution was collected for analysis. Any antibodies that were transcytosed into the outer chamber were excluded from the samples, avoiding any contamination of the recycling assay by FcRn-mediated transcytosis. The integrity of the monolayer was evaluated at the end of each experiment and data from compromised monolayers was excluded from analysis.

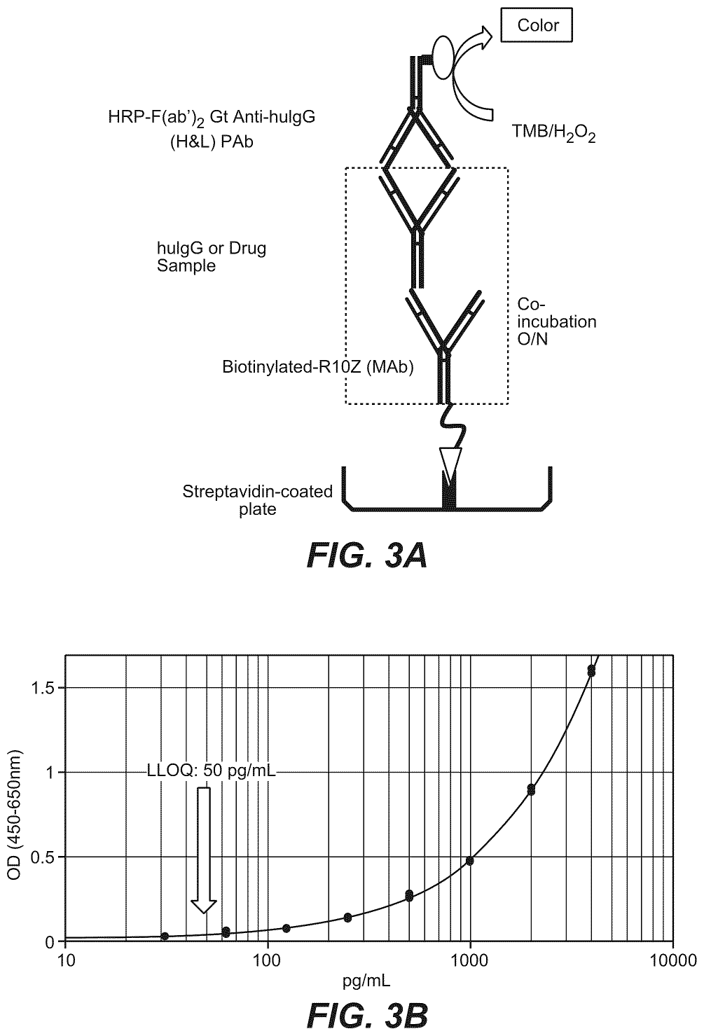

As the quantity of recycled antibodies released from cells in a 96-well assay format is low, a sensitive human IgG-specific enzyme-linked immunosorbent assay (ELISA) was developed. This sandwich ELISA used a biotinylated mouse anti-human IgG-Fc as capture, HRP-conjugated goat anti-human Fab as detection, and TMB solution for color development (FIG. 3A), the lower limit of quantification (LLOQ) achieved 50 pg/mL which allows quantification of sub-nanomolar concentrations of the recycling outputs (FIG. 3B).

### Example 2: Recycling Output of Humanized IgG1 Antibodies Is Largely Dependent Upon FcRn Mediated Mechanism in Cells Expressing FcRn

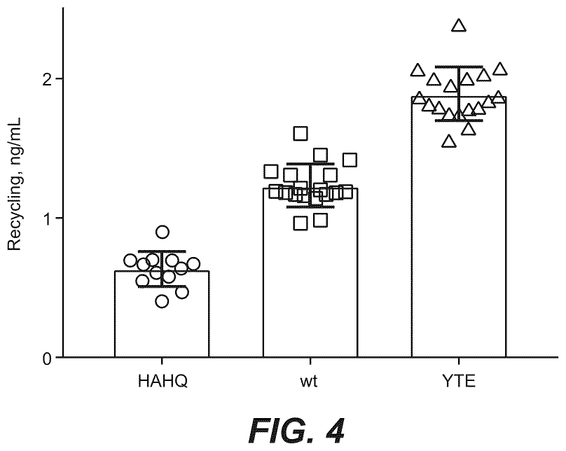

In order to demonstrate the involvement of FcRn mediated mechanism in the recycling assay, a humanized IgG1 antibody—anti-herpes simplex virus glycoprotein D (anti-gD) wildtype (WT) was compared with two variants carrying Fc mutation that alters Fc-FcRn binding affinity. One of the variants anti-gD HAHQ carries H310A/H435Q mutations that abolish FcRn binding, and the other variant anti-gD YTE carries FcRn binding enhancing mutations M252Y/S254T/T256E. The recycling output of anti-gD HAHQ was 0.6 ng/mL, significantly lower than that of anti-gD WT(1.2 ng/mL), whereas anti-gD YTE had an elevated recycling output (1.9 ng/mL) (FIG. 4). These results suggested that the major mechanism reflected by the recycling output in this system is mediated by FcRn, and supports the use of this assay for evaluating FcRn-mediated IgG homeostasis.

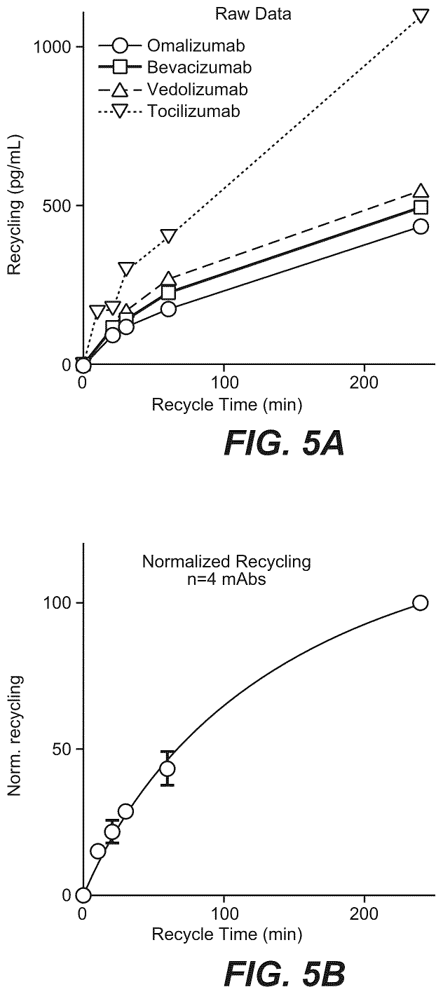

Next the kinetics of recycling measurements was evaluated by using a group of conventional humanized therapeutic antibodies, including omalizumab, bevacizumab, vedolizumab and tocilizumab. The recycling samples were taken at various time points during the chase step, ranging from 20 minutes to 4 hours. For each individual molecule, the cumulative increase of recycling was observed, with lowest recycling output at 20 minutes and highest output at 4 hours (FIG. 5A). Three of the evaluated antibodies demonstrated a maximum recycled amount of approximately 0.4-0.5 ng/ml, while tocilizumab had higher recycling values, reaching 1.09 ng/ml by 4 hours. Despite the difference in recycling outputs among the four molecules, the kinetics of recycling is similar, showing little variations throughout the time course of the chase step (FIG. 5B; recycling outputs were normalized to the 4-hour data point of the same molecule; data tabulated in Table 1B). The ranking order of recycling outputs among the four molecules was further examined at each time points with normalization to the recycling outputs of bevacizumab. Both the ranking order and the normalized recycling outputs were similar across the four time points (Table 1A, normalized to Bevacizumab), suggesting that the relativity of recycling outputs is consistent regardless of chase time. The coefficient of variation (CV) across the four time points (20, 30, 60, and 240 min) was less than 20% for each molecule, suggesting the relative recycling output among antibodies with different absolute recycling values was consistent over the 4-hour chase. Accordingly, 4 hours in the pulse step was selected as the standard assay procedure for this system due to the higher recycling outputs which gives more confidence on quantification, and it was decided to use bevacizumab as an internal control for normalization of recycling outputs measured with other test molecules.

TABLE 1A
Recycling output normalized to Bevacizumab at each time point.

| Recycle | | | | |
|---|---|---|---|---|
| Time (min) | Omalizumab | Bevacizumab | Vedolizumab | Tocilizumab |
| 20.00 | 0.82 | 1.00 | 1.18 | 1.51 |
| 30.00 | 0.87 | 1.00 | 1.22 | 2.13 |
| 60.00 | 0.77 | 1.00 | 1.20 | 1.77 |
| 240.00 | 0.88 | 1.00 | 1.11 | 2.22 |
| Mean | 0.83 | 1.00 | 1.18 | 1.91 |
| SD | 0.05 | 0.00 | 0.05 | 0.33 |
| CV % | 6.26 | 0.00 | 4.00 | 17.10 |

TABLE 1B
Averaged recycling outputs at earlier time points normalized to the maximum
value at 240 minutes for each antibody. Mean and SD of the normalized
outputs from the four antibodies were calculated at each time point.

| Recycle | Omalizumab | Bevacizumab | Vedolizumab | Tocilizumab | Mean | SD |
|---|---|---|---|---|---|---|
| Time (min) | (% of max) | (% of max) | (% of max) | (% of max) | (% of max) | (% of max) |
| 20 | 21.7 | 23.4 | 25.0 | 16.0 | 21.5 | 3.9 |
| 30 | 27.7 | 28.1 | 30.9 | 27.1 | 28.4 | 1.7 |
| 60 | 40.0 | 46.0 | 49.6 | 36.7 | 43.1 | 5.8 |
| 240 | 100 | 100 | 100 | 100 | 100.0 | 0.0 |

### Example 3: Correlation of Transcytosis/Recycling (T/R) Ratio with in Vivo Brain Serum Ratio in Cynomolgus

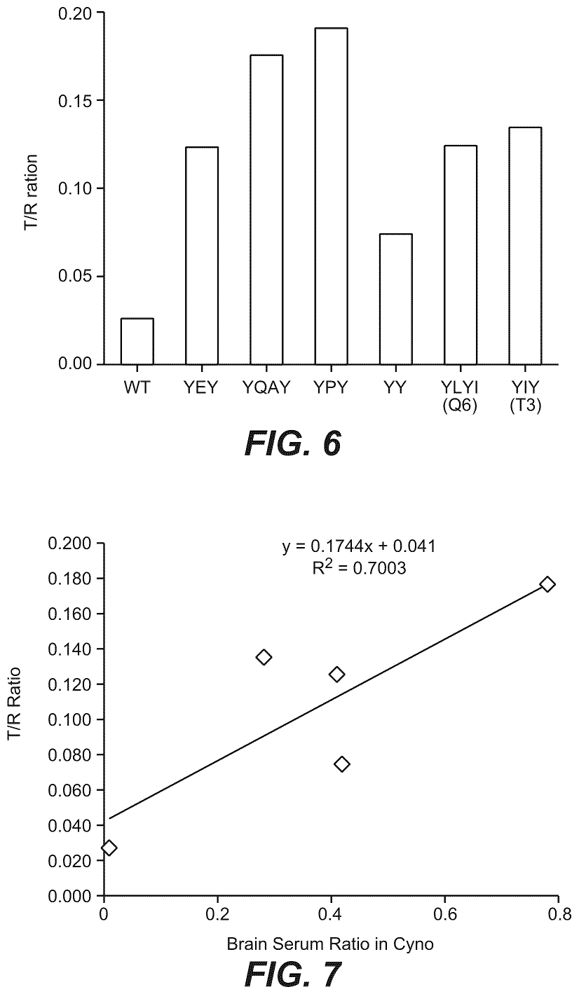

A panel of 7 anti-BACE1 variants, including the wildtype (WT) molecule and six variants, were tested in the transcytosis/recycling dual assay. The anti-BACE1 antibodies are described further in W02020/132230A2. The six variants carry mutations on their Fc region that significantly enhanced binding affinity to FcRn at neutral pH, with Kd at pH 7.4 lowered by 13.3-78.1-fold (Table 1). The higher binding affinity to FcRn would lead to more protection of mAbs, guiding mAbs more towards FcRn mediated recycling and transcytosis pathways. Compared with the WT molecules, the pH 7.4 variants showed significant increase in both recycling and transcytosis outputs. This finding is likely attributable to the enhancement of mAb internalization through FcRn receptor mediated uptake for pH 7.4 variants. Furthermore, the ratio between transcytosis and recycling was significantly increased among pH 7.4 variants. The T/R ratio for WT molecule was 0.026, whereas the ratio ranged from 0.074 to 0.191 for pH 7.4 variants (Table 2 and FIG. 6), suggesting the mutations introduced bias towards transcytosis pathway that may promote antibody transport across the cell layer.

TABLE 2

| | | Kd at 7.4 | Recycling | Transcytosis | |
|---|---|---|---|---|---|
| Variant | Mutations | (nM) | (ng/mL) | (ng/mL) | T/R ratio |
| WT | — | 5000 | 5.59 | 0.148 | 0.026 |
| YEY | M252Y; N286E; N434Y | 288 | 306.5 | 38.1 | 0.124 |
| YQAY | M252Y; T307Q; Q311A; N434Y | 153 | 274.6 | 48.4 | 0.176 |
| YPY | M252Y; V308P; N434Y | 64 | 378.4 | 72.3 | 0.191 |
| YY | M252Y; N434Y | 376 | 285.4 | 21.2 | 0.074 |
| YLYI (Q6) | M252Y; M428L; N434Y; Y436I | 181 | 423.5 | 52.9 | 0.125 |
| YIY (T3) | M252Y; Q311I; N434Y | 328 | 343.8 | 46.3 | 0.135 |

The potential correlation between the assay outputs and in vivo brain penetration results from cyno studies was then analyzed (cyno data available for 5 molecules; Table 3). Transcytosis, or recycling outputs alone did not have a significant correlation with the brain serum ratio. However, the T/R ratio demonstrated a very strong correlation with the brain serum ratio (pearson's correlation coefficient 0.837). Higher T/R ratio indicates a biased intracellular pathway in favor of transcytosis and correlated a higher brain penetration behavior in vivo (FIG. 7). Therefore, the T/R ratio is a meaningful biological parameter for the prediction of brain penetration capabilities of therapeutic candidates.

TABLE 3
Cyno data for five molecules, obtained
as described in WO2020/132230A2

| Mutation | Serum AUC | Brain serum ratio - day 2 | Kd at 7.4 |
|---|---|---|---|
| WT | 1 | 0.01 | 5000 |
| YQAY | 0.24 | 0.78 | 153 |
| YY | 0.47 | 0.42 | 376 |
| YLYI (Q6) | 0.17 | 0.41 | 181 |
| YIY (T3) | 0.2 | 0.28 | 328 |

### Example 3: Correlation of in Vitro Recycling Output with in Vivo Clearance of mAbs in Humans

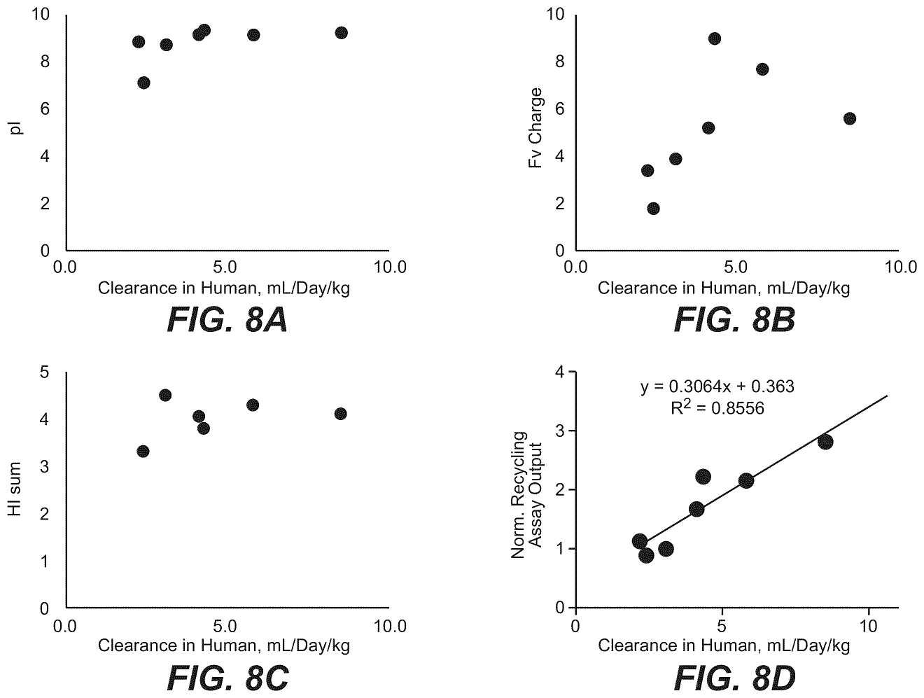

Seven therapeutic antibodies, including five marketed drugs and two previously under development, were tested and evaluated for potential correlation with in vivo clearance in humans. The seven molecules are humanized antibodies in IgG1/kappa isotype and reflect diversified properties, including mechanism of action, target property and route of administration, but none carry Fc mutations for modifying PK properties or FcRn interactions. Based on the drug's prescribing information and previous publication, the clearance values cover a range from 2.2 to 8.5 mL/day/kg. The observed discrepancy in nonspecific clearance among similar antibody isotype has been proposed to result from molecular properties, such as pI, charge or hydrophobicity (Hotzel, I. et al., (2012), MAbs, 4(6), 753-760). Using an established sequence-based algorithm, we calculated molecular parameters including (i) pI value, (ii) net Fv charge at pH 5.5, and (iii) sum of hydrophobicity index values (HIsum) on CDRs L1, L3, H2, and H3 (Sharma, V. K. et al., (2014), PNAS, 111(52), 18601-18606). Extreme values of these parameters have been shown to be indicative of antibodies with faster clearance, however, consistent with the previous findings, no apparent trend of correlations between these parameters with clearance were observed (FIGS. 8A-8C; Table 4). To explore correlation between recycling and clearance, the seven molecules were evaluated in the recycling assay to obtain a quantitative measurement of the FcRn mediated recycling process. The recycling output of each molecule, measured by the concentration of antibodies returned to the inner chamber, was normalized to the recycling value of bevacizumab running on the same 96-well transwell plate as a control. The results of the recycling assay with these molecules, along with their published clearance in human are shown in Table 4. All seven molecules demonstrated measurable recycling outputs, with molecules of faster clearance values demonstrating a higher recycling outputs. The recycling output showed a strong positive correlation with clearance in humans (R²=0.856 for recycling; FIG. 8D). The average intra-assay precision is 10.7% and inter-assay precision is 12.3%. This result suggests recycling as a promising assay for the prediction of PK behavior of therapeutic antibodies or candidates in development.

TABLE 4
Normalized recycling outputs and calculated PI, Fv and HIsum of evaluated antibodies

| | | | Clearance in | Recycling | | | |
|---|---|---|---|---|---|---|---|
| | | Route of | Human, | 4 hrs, | | Fv | |
| mAb | Target | admin | mL/Day/Kg | normalized | PI | charge | Hisum |
| Bevacizumab | VEGF | iv | 3.1 | 1 | 8.75 | 3.9 | 4.48 |
| Omalizumab | IgE | sc | 2.4 | 0.88 | 7.15 | 1.8 | 3.29 |
| Vedolizumab | Integrin | iv | 2.2 | 1.11 | 8.85 | 3.4 | 2.97 |
| | (α4β7) | | | | | | |
| Tocilizumab | IL6R | sc | 4.3 | 2.22 | 9.35 | 9 | 3.79 |
| Adalimumab | TNFα | sc | 4.1 | 1.68 | 9.15 | 5.2 | 4.04 |
| mAbX | Not | sc | 5.8 | 2.16 | 9.15 | 7.7 | 4.28 |
| | disclosed | | | | | | |
| mAbY | Not | iv | 8.5 | 2.81 | 9.25 | 5.6 | 4.1 |
| | disclosed | | | | | | |

iv—intravenous;
sc—subcutaneous

The seven evaluated antibodies, each with conventional Fc region, demonstrated a notable correlation between recycling output and clearance in humans, regardless of diverse Fab region properties. IgGs with identical Fc regions can vary widely in Pk properties, which has been attributed to the Fv charge of the Fab arm, may impact interaction with FcRn, and may impact fluid phase pinocytosis during internalization. In this study, following normalization to maximum value at 4 hours for each molecule, there was little variation between the recycling kinetics curves among the four humanized therapeutic antibodies tested (omalizumab, bevacizumab, vedolizumab, and tocilizumab). The rate of the recycling (slope at each time point) was identical despite tocilizumab exhibiting a much higher recycling output value, ˜two-fold greater than that of the other three antibodies at 4 hours. Without wishing to be bound by theory, the similar recycling kinetics may suggest that, for mAbs with a conventional Fc region, FcRn-mediated transport through the recycling pathway may be similar such that differences in the measured amount of recycling output may result from mechanisms upstream to the FcRn-mediated transport. These four antibodies have comparable binding affinities to FcRn at pH 6.0, but tocilizumab has a higher calculated PI value and a higher Fv charge. This may explain the high recycling value and faster clearance of tocilizumab, because the positive charge may enhance nonspecific binding through electrostatic interactions with the anionic heparan sulfate proteoglycans at the cell surface. These interactions could possibly increase internalization of tocilizumab through fluid phase pinocytosis. Previous work has shown that a FcRn-independent mechanism may play a role in regulating PK behavior of mAbs sharing identical Fc but with different Fab regions.

Without wishing to be bound by theory, given that recycling has been considered as a salvage mechanism that rescues IgG from lysosomal degradation and escorts it back to the blood, it was initially expected that a high recycling output may inversely correlate with clearance rate. Surprisingly, the opposite was observed: higher recycling output corresponded with faster clearance. Without wishing to be bound by theory, this positive correlation between clearance and FcRn-mediated recycling may relate to the fraction of the antibody that undergoes elimination mechanisms during each internalization event. The recycling process takes antibodies from circulation and returns them back to the same compartment, but does not affect the net amount of serum concentration as the sole factor by itself. However, it has been demonstrated previously that the recycling event can be accompanied by a small-fraction loss of antibodies, which was attributed to the degradation in lysosomes (Grevys et al., 2018). In the presently described transwell culturing system, transcytosis may also serve as an eliminating pathway that guides antibodies away from recycling. Without being bound by theory, it is speculated that such loss (through degradation and transcytosis) for each recycling event may reduce the recycled amount back to the circulation. Therefore, for the high recycling molecules, the loss is exaggerated and may contribute to a faster reduction in serum concentration, and a faster measured clearance. Interestingly, the observation seems to be reversed for the FcRn binding variant group, demonstrating a trend of negative correlation between recycling outputs and clearance for the Fc-engineered anti-gD variants. The YTE mutation, which demonstrated slower clearance, had the higher recycling output compared with WT (See Example 2). Without being bound by theory, the much-enhanced binding to FcRn may have become the dominant biological factor, which may lead to increased level of protection and reduced degradation in lysosomes that extended the antibody's serum half-life. To explain the discrepancy between conventional and Fc-engineered mAbs, it is possible that the dominant factors that affect PK behavior vary between the two scenarios depending on the design of the molecules. Thus, biochemical properties and Fc-engineering of the antibodies should be considered when interpreting the results from this in vitro system.

The seven mAbs evaluated indicate the recycling assay described herein was superior to other models using pI, charge, or hydrophobicity parameters of the antibody as a predictor of PK properties of mAbs without Fc modifications, in particular clearance from circulation in humans. The assay design uses a three-compartment in vitro model system (inner chamber, cell, and outer chamber) to mimic the in vivo situation (blood compai intent, vesicular endothelial cells, and tissue compartment), and incorporates multiple biological events that may affect cellular metabolism of antibodies. The recycling assay provides a holistic evaluation of multiple biological factors that may affect cellular metabolism of antibodies, and therefore may better predict one or more PK properties compared to other published assays that consider only a single or a subset of factors, e.g. nonspecific binding, binding to extracellular matrix, and FcRn interactions.

Further, without wishing to be bound by theory, the neutral pH used in both transwell chambers may be key to a successful prediction of PK properties, as this is more physiologically relevant to the in vivo situation where antibodies are captured and released at neutral pH at the cell surface. This may explain the limited application of other FcRn-mediated cell-based assays where acidic buffer was used in loading the antibodies into the cells to take advantage of the enhanced FcRn binding in acidic pH (Chung et al., (2018), J Immunol Methods, 462, 101-105; Jaramillo et al., 2017, MAbs, 9(5), 781-791; Sockolosky, J T et al., 2012, PNAS, 109(40), 16095-16100; Ying et al., 2015, MAbs, 7(5), 922-930). Some of these methods predicted clearance or half-life of Fc engineered molecules with enhanced FcRn binding, but had limited ability to predict PK behavior of conventional mAbs in which changes to FcRn bindings are more subtle. Further, cell polarization may be considered an important factor because both recycling and transcytosis are inherently polarized and are at least in part carried by independent processes at the apical and basolateral surface of polarized endothelial cells (Dickinson et al., 1999, J Clin Invest, 104(7), 903-911; Tzaban, S. et al., 2009, J Cell Biol, 185(4), 673-684). The assay described herein utilizing a transwell cell culture system may allow for better polarization and separation of recycling from the transcytosis component, which may be more accurate in evaluating conventional mAbs, compared to other recycling assays performed on a regular cell culture plate (Grevys, A. Nat Commun, 9(1) 621).

All publications, patents and other references cited herein are incorporated by reference in their entirety into the present disclosure.

What is claimed is:

1. A method, comprising determining the recycling of a plurality of molecules, wherein the determining comprises:
introducing the plurality of molecules into a first chamber, wherein:
the first chamber is separated from a second chamber by a cell layer, and
the first and second chambers comprise an aqueous solution;
incubating the plurality of molecules in the first chamber;
replacing the aqueous solution in both the first and second chambers; and
measuring the amount of the plurality of molecules that is released from the cell layer into the first chamber;
wherein the aqueous solution is at physiological pH, and the cell layer comprises cells that express a receptor that mediates molecular transport.

2. The method of claim 1, wherein the cell layer comprises a cell monolayer.

3. The method of claim 1 or 2, wherein the receptor that mediates molecular transport is a transferrin receptor, an Fc receptor, megalin, or cubulin.

4. The method of any one of claims 1 to 3, wherein the receptor that mediates molecular transport is a neonatal Fc receptor (FcRn).

5. The method of any one of claims 1 to 3, wherein the cells are Madin-Darby Canine Kidney (MDCK) cells.

6. The method of any one of claims 1 to 4, wherein measuring the amount of the plurality of molecules that is released from the cell layer comprises the use of an enzyme-linked immunosorbent assay (ELISA), liquid-scintillation counting (LSC), quantitative PCR, a fluorescence reader system, or mass spectrometry.

7. The method of any one of claims 1 to 5, wherein the plurality of molecules are Fc-containing molecules.

8. The method of any one of claims 1 to 5, wherein the plurality of molecules are antibodies.

9. The method of claim 4, wherein the FcRn is selected from the group consisting of human RcRn, mouse FcRn, rat FcRn, and cynomolgus FcRn.

10. The method of any one of claims 1 to 9, further comprising measuring the transcytosis of the plurality of molecules across the cell layer.

11. The method of claim 10, wherein measuring the transcytosis comprises:
after the aqueous solution has been replaced in both the first and second chambers, measuring the amount of the plurality of molecules in the second chamber.

12. The method of any one of claims 1 to 11, comprising incubating the plurality of molecules in the first chamber in the presence of an agent, and determining whether the agent affects the recycling of the plurality of molecules.

13. A method of determining the tissue penetrance of a plurality of molecules, comprising:
a) introducing the plurality of molecules into a first chamber, wherein the first chamber is separated from a second chamber by a cell layer, and the first and second chambers comprise an aqueous solution;
wherein the aqueous solution is at physiological pH, and the cell layer comprises cells that express a receptor that mediates molecular transport;
b) measuring the amount of the plurality of molecules that is recycled from the first chamber into the cell layer and back to the first chamber;
c) measuring the amount of the plurality of molecules that is transcytosed from the first chamber to the second chamber; and
d) determining the tissue penetrance of the plurality of molecules based on the ratio of transcytosed to recycled molecules.

14. The method of claim 13, wherein measuring the plurality of molecules that is recycled comprises:
after introducing the plurality of molecules into the first chamber, incubating the plurality of molecules in the first chamber;
replacing the aqueous solution in both the first and second chambers; and
measuring the amount of the plurality of molecules that is released from the cell layer into the first chamber.

15. The method of claim 13 or 14, wherein measuring the plurality of molecules that is transcytosed comprises:
after introducing the plurality of molecules into the first chamber, incubating the plurality of molecules in the first chamber;
replacing the aqueous solution in both the first and second chambers; and
measuring the amount of the plurality of molecules that is released from the cell layer into the second chamber.

16. The method of any one of claims 13 to 15, wherein the cell layer comprises a cell monolayer.

17. The method of any one of claims 13 to 16, wherein the receptor that mediates molecular transport is a transferrin receptor, an Fc receptor, megalin, or cubulin.

18. The method of any one of claims 13 to 17, wherein the receptor that mediates molecular transport is a neonatal Fc receptor (FcRn).

19. The method of any one of claims 13 to 18, wherein the cells are Madin-Darby Canine Kidney (MDCK) cells.

20. The method of any one of claims 13 to 19, wherein measuring the amount of the plurality of molecules that is recycled and measuring the amount of the plurality of molecules that is transcytosed independently comprise the use of an enzyme-linked immunosorbent assay (ELISA), liquid-scintillation counting (LSC), quantitative PCR, a fluorescence reader system, mass spectrometry, or any combinations thereof.

21. The method of any one of claims 13 to 20, wherein the plurality of molecules are Fc-containing molecules.

22. The method of any one of claims 13 to 21, wherein the plurality of molecules are antibodies.

23. The method of claim 22, wherein the antibody is an anti-IgE antibody, an anti-VEGF antibody, an anti-integrin antibody, an anti-IL-6 antibody, an anti-TNFa antibody, an anti-BACE1 antibody, or an anti-gD antibody.

24. The method of any one of claims 13 to 23, wherein the tissue penetrance is brain penetrance.

25. The method of any one of claims 13 to 24, comprising incubating the plurality of molecules in the first chamber in the presence of an agent, and determining whether the agent affects the tissue penetrance of the plurality of molecules.

26. A method of determining a pharmacokinetic (PK) parameter of a plurality of molecules, comprising:
a) introducing the plurality of molecules into a first chamber, wherein the first chamber is separated from a second chamber by a cell layer, and the first and second chambers comprise an aqueous solution;
wherein the aqueous solution is at physiological pH, and the cell layer comprises cells that express a receptor that mediates molecular transport;
b) measuring the amount of the plurality of molecules that is recycled from the first chamber into the cell layer and back to the first chamber; and
c) determining the PK parameter based on the amount of the plurality of molecules that is recycled.

27. The method of claim 26, wherein measuring the plurality of molecules that is recycled comprises:
after introducing the plurality of molecules into the first chamber, incubating the plurality of molecules in the first chamber;
replacing the aqueous solution in both the first and second chambers; and
measuring the amount of the plurality of molecules that is released from the cell layer into the first chamber.

28. The method of claim 26 or 27, wherein the cell layer comprises a cell monolayer.

29. The method of any one of claims 26 to 28, wherein the cells express a heterologous FcRn.

30. The method of any one of claims 26 to 29, wherein the cells are Madin-Darby Canine Kidney (MDCK) cells.

31. The method of any one of claims 26 to 29, wherein measuring the amount of the plurality of molecules that is recycled comprises the use of an enzyme-linked immunosorbent assay (ELISA), liquid-scintillation counting (LSC), quantitative PCR, a fluorescence reader system, or mass spectrometry.

32. The method of any one of claims 26 to 31, wherein the plurality of molecules are Fc-containing molecules.

33. The method of any one of claims 26 to 32, wherein the plurality of molecules are antibodies.

34. The method of claim 33, wherein the antibody is an anti-IgE antibody, an anti-VEGF antibody, an anti-integrin antibody, an anti-IL-6 antibody, an anti-TNFa antibody, an anti-BACE1 antibody, or an anti-gD antibody.

35. The method of any one of claims 26 to 34, wherein the PK parameter is a measure of in vivo clearance, volume of distribution, area under the curve (AUC), or in vivo half-life of the plurality of molecules.

36. The method of any one of claims 26 to 35, comprising incubating the plurality of molecules in the first chamber in the presence of an agent, and determining whether the agent affects the PK parameter of the plurality of molecules.

37. An assay system, comprising:
a) a first chamber and a second chamber, wherein each chamber comprises aqueous solution at physiological pH;
b) a cell layer separating the first and second chamber, wherein the cell layer can mediate recycling of a molecule from the first chamber, into the cell layer, and back into the first chamber;
c) a detector for detecting the presence of a molecule in the first chamber;
wherein the assay system is configured to determine the recycling of a plurality of molecules, wherein the determining comprises:
introducing the plurality of molecules into the first chamber;
incubating the plurality of molecules in the first chamber;
replacing the aqueous solution in both the first and second chambers; and
measuring the amount of the plurality of molecules that is released from the cell layer into the first chamber.

38. The assay system of claim 37, wherein:
the assay system further comprises a detector for detecting the presence of a molecule in the second chamber;
the cell layer can mediate the transcytosis of a molecule from the first chamber to the second chamber; and
wherein the assay system is configured to determine the transcytosis of a plurality of molecules across the cell layer, wherein determining transcytosis comprises, after the aqueous solution has been replaced, measuring the amount of the plurality of molecules in the second chamber.

39. The assay system of claim 37 or 38, wherein the first and second chambers are components of a 96-well trans-well plate.

40. A kit, comprising the assay system of any one of claims 37 to 39, and instructions for use.


SEQUENCE LISTING (8 sequences)

SEQ ID NO: 1 - protein, 342 aa, Homo sapiens
AESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

SEQ ID NO: 2 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - protein, 344 aa, Mus sp.
SETRPPLMYHLTAVSNPSTGLPSFWATGWLGPQQYLTYNSLRQEADPCGAWMWENQVSWYWEKETTDLKSKEQLFLEALKTLEKILNGTYTLQGLLGCELASDNSSVPTAVFALNGEEFMKFNPRIGNWTGEWPETEIVANLWMKQPDAARKESEFLLNSCPERLLGHLERGRRNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSSARSSVPVVGIVLGLLLVVVAIAGGVLLWGRMRSGLPAPWLSLSGDDSGDLLPGGNLPPEAEPQGANAFPATS

SEQ ID NO: 4 - protein, 119 aa, Mus sp.
MARSVTLVFLVLVSLTGLYAIQKTPQIQVYSRHPPENGKPNILNCYVTQFHPPHIEIQMLKNGKKIPKVEMSDMSFSKDWSFYILAHTEFTPTETDTYACRVKHDSMAEPKTVYWDRDM

SEQ ID NO: 5 - protein, 344 aa, Rattus sp.
AEPRLPLMYHLAAVSDLSTGLPSFWATGWLGAQQYLTYNNLRQEADPCGAWIWENQVSWYWEKETTDLKSKEQLFLEAIRTLENQINGTFTLQGLLGCELAPDNSSLPTAVFALNGEEFMRFNPRTGNWSGEWPETDIVGNLWMKQPEAARKESEFLLTSCPERLLGHLERGRQNLEWKEPPSMRLKARPGNSGSSVLTCAAFSFYPPELKFRFLRNGLASGSGNCSTGPNGDGSFHAWSLLEVKRGDEHHYQCQVEHEGLAQPLTVDLDSPARSSVPVVGIILGLLLVVVAIAGGVLLWNRMRSGLPAPWLSLSGDDSGDLLPGGNLPPEAEPQGVNAFPATS

SEQ ID NO: 6 - protein, 119 aa, Rattus sp.
MARSVTVIFLVLVSLAVVLAIQKTPQIQVYSRHPPENGKPNFLNCYVSQFHPPQIEIELLKNGKKIPNIEMSDLSFSKDWSFYILAHTEFTPTETDVYACRVKHVTLKEPKTVTWDRDM

SEQ ID NO: 7 - protein, 342 aa, Macaca fascicularis
AESHLSLLYHLTAVSSPAPGTPAFWVSGWLGPQQYLSYDSLRGQAEPCGAWVWENQVSWYWEKETTDLRIKEKLFLEAFKALGGKGPYTLQGLLGCELSPDNTSVPTAKFALNGEEFMNFDLKQGTWGGDWPEALAISQRWQQQDKAANKELTFLLFSCPHRLREHLERGRGNLEWKEPPSMRLKARPGNPGFSVLTCSAFSFYPPELQLRFLRNGMAAGTGQGDFGPNSDGSFHASSSLTVKSGDEHHYCCIVQHAGLAQPLRVELETPAKSSVLVVGIVIGVLLLTAAAVGGALLWRRMRSGLPAPWISLRGDDTGSLLPTPGEAQDADSKDINVIPATA

SEQ ID NO: 8 - protein, 119 aa, Macaca fascicularis
MSPSVALAVLALLSPSGLEAIQRTPKIQVYSRHPPENGKPNFLNCYVSGFHPSDIEVDLLKNGEKMGKVEHSDLSFSKDWSFYLLYYTEFTPNEKDEYACRVNHVTLSGPRTVKWDRDM